SEQUENCE LISTING (2 sequences)

SEQ ID NO: 1 - protein, 55 aa, Homo sapiens
MNYPLTLEMDLENLEDLFWELDRLDNYNDTSLVENHLCPATEGPLMASFKAVFVP

SEQ ID NO: 2 - DNA, 186 bp, Homo sapiens
atgaagcgcttcctcttcctcctactcaccatcagcctcctggttatggtacagatacaaactggactctcaggacaaaacgacaccagccaaaccagcagcccctcagcatccagcagcatgagcggaggcattttccttttcttcgtggccaatgccataatccacctcttctgcttcagttga

(19) United States
(12) Patent Application Publication
(10) Pub. No.: US 2014/0256918 A1
(43) Pub. Date: Sep. 11, 2014

(54) METHOD FOR IMMOBILIZING MEMBRANE PROTEINS ON SURFACES

(71) Applicant: SANOFI, Paris (FR)

(72) Inventors: Ruiyin Chu, Westborough, MA (US); David James Reczek, Sudbury, MA (US)

(21) Appl. No.: 14/158,056

(22) Filed: Jan. 17, 2014

Related U.S. Application Data
(63) Continuation-in-part of application No. 13/745,377, filed on Jan. 18, 2013.
Application No. 13/745,377 is a continuation-in-part of application No. PCT/US13/22280, filed on Jan. 18, 2013.

(51) Int. Cl. G01N 33/543

(52) U.S. Cl. CPC G01N 33/54353
USPC 530/400; 530/402; 530/409

(57) ABSTRACT

Disclosed herein are methods for immobilizing membrane proteins or membrane protein complexes on analytical surfaces, which in some aspects comprise: obtaining a membrane protein or membrane protein complex comprising a capture moiety; immobilizing the membrane protein or membrane protein complex on the analytical surface by means of the capture moiety; and stabilizing at least one of the secondary, tertiary, or quaternary structures of the immobilized membrane protein or membrane protein complex by crosslinking with a crosslinking reagent. Also disclosed are analytical surfaces, which in some aspects comprise: a membrane protein or membrane protein complex comprising a capture moiety, wherein the membrane protein or membrane protein complex is immobilized on the analytical surface by means of the capture moiety, and wherein at least one of the secondary, tertiary, or quaternary structures of the membrane protein or membrane protein complex is stabilized by crosslinking.

This application claims the benefit of priority under 35 U.S.C. §120 and is a continuation-in-part of U.S. application Ser. No. 13/745,377, filed Jan. 18, 2013, and a continuation-in-part of International Application No. PCT/US13/22280, filed Jan. 18, 2013, the disclosures of each of which are explicitly incorporated herein by reference in their entirety.

### BACKGROUND OF THE INVENTION

Membrane proteins such as receptors and ion channels are key regulators of cellular function. Membrane proteins account for up to two thirds of known drugable targets, highlighting their critical pharmaceutical importance. The G protein-coupled receptors (GPCRs) are the largest, most versatile, group of membrane receptors and also the most pharmaceutically important, accounting for over 50% of all human drug targets and acting as therapeutic targets for a wide range of disease conditions including cancer, cardiovascular, metabolic, CNS and inflammatory diseases.

Surface plasmon resonance (SPR) biosensor-based assays, such as Biacore assays, have been used to study protein interactions in real time without labeling. Biacore pioneered commercial SPR biosensors offering a unique technology for collecting high-quality, information-rich data from biomolecular binding events. Since the release of the first instrument in 1990, researchers around the world have used Biacore's optical biosensors to characterize binding events with samples ranging from proteins, nucleic acids, small molecules to complex mixtures, lipid vesicles, viruses, bacteria, and eukaryotic cells. However, the application of SPR assays to study membrane-associated systems such as GPCRs is still in its infancy.

The challenges of studying membrane-associated proteins with optical biosensors are 2-fold. First, most membrane proteins, such as GPCRs, are expressed at low levels and are unstable when extracted from the hydrophobic cell membrane environment. This makes it difficult to immobilize these receptors onto the sensor surface while maintaining high levels of activity. Second, the ligands for most membrane proteins have low molecular weights (e.g., histamine [111 Da] and serotonin [176 Da] for GPCRs). This places an added burden on surface plasmon resonance (SPR) biosensor technology, which is mass based.

One approach, developed by Karlsson and Löfås, is to immobilize a purified receptor onto the sensor surface and then reconstitute a membrane environment on the surface (Anal. Biochem., 2002, 300(2):132-8). Myszka and co-workers extended this method by showing that it was possible to capture receptors out of crude preparations and directly study the binding of antibodies (Anal. Biochem., 2006, 355(1):132-9). However, with these methods, the lipid bilayer reconstituted on the chip surface is unstable and cannot be regenerated. Therefore, this method is rarely used.

Another approach is to engineer membrane proteins specifically for immobilization. For example, in studies by Myszka and co-workers, GPCRs were engineered using point mutations, yielding improved thermostability and conformational homogeneity (Anal. Biochem., 2011, 409(2):267-72); additionally, the engineered GPCRs retained activity after immobilization onto Biacore sensor chip surfaces. However, this approach is time consuming and a stable engineered molecule may not always be achievable. In addition, this method requires pre-activation of nickel-nitrilotriacetic acid (Ni-NTA) SPR surfaces by a non-specific amine coupling reagent prior to capturing the purified receptor. Therefore, any contaminated protein can be immobilized on the activated NTA surface via this process as long as it contains a primary amine group. This yields low binding activities for the surface-immobilized target protein. Furthermore, after binding, the surface can only be regenerated with a low affinity binder, which makes it impossible for analyzing large molecules such as antibodies.

### SUMMARY OF THE INVENTION

The present invention provides certain advantages and advancements over the prior art. In particular, the present disclosure provides methods of stabilizing captured membrane proteins, for example, membrane receptors such as G-protein-coupled receptors (GPCRs), on analytical surfaces, for example, surface plasmon resonance surfaces such as Biacore sensor chip surfaces, by limited chemical crosslinking. This limited chemical crosslinking is efficient and reliable. It enables high quality kinetics assays of membrane proteins via the same methods that are conventionally used for soluble proteins. Also described here are methods for capturing and stabilizing membrane protein complexes, for example, virus like particles (VLPs), on analytical surfaces for binding kinetics assays by limited chemical crosslinking.

In one aspect, the present disclosure provides methods for immobilizing a membrane protein or membrane protein complex on an analytical surface, the methods comprising: (a) obtaining a membrane protein or membrane protein complex comprising a capture moiety; (b) immobilizing the membrane protein or membrane protein complex on the analytical surface by means of the capture moiety; and (c) stabilizing at least one of the secondary, tertiary, or quaternary structures of the immobilized membrane protein or membrane protein complex by crosslinking the immobilized membrane protein or membrane protein complex with a crosslinking reagent.

In another aspect, the present disclosure provides methods for immobilizing a membrane protein or membrane protein complex on an analytical surface, the methods comprising: (a) obtaining a membrane protein or membrane protein complex modified with a capture moiety; (b) coupling to the analytical surface an antibody specific for the capture moiety using a coupling reagent; (c) immobilizing the membrane protein or membrane protein complex on the analytical surface by means of interaction between the antibody and the capture moiety; and (d) stabilizing at least one of the secondary, tertiary, or quaternary structures of the immobilized membrane protein or membrane protein complex by crosslinking the immobilized membrane protein or membrane protein complex with a crosslinking reagent.

In another aspect, the present disclosure provides analytical surfaces comprising: a membrane protein or membrane protein complex comprising a capture moiety, wherein the membrane protein or membrane protein complex is immobilized on the analytical surface by means of the capture moiety, and wherein at least one of the secondary, tertiary, or quaternary structures of the membrane protein or membrane protein complex is stabilized by crosslinking.

In another aspect, the present disclosure provides analytical surfaces comprising: (a) a membrane protein or membrane protein complex comprising a capture moiety, and (b) an antibody specific for the capture moiety, wherein the antibody is coupled to the analytical surface using a coupling reagent, wherein the membrane protein or membrane protein complex is immobilized on the analytical surface by means of interaction between the antibody and the capture moiety, and wherein at least one of the secondary, tertiary, or quaternary structures of the membrane protein or membrane protein complex is stabilized by crosslinking.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, in which:

FIG. 1 shows a conventional peptide-based Biacore assay for antibodies against hCXCR5. First an anti-mouse antibody is amine coupled on a CM5 chip surface, then a mouse anti-hCXCR5 mAb is captured. A hCXCR N-terminal peptide (MNYPLTLEMD LENLEDLFWE LDRLDNYNDT SLVENHLCPA TEGPLMASFK AVFVP) (SEQ ID NO: 1) is injected over the captured anti-hCXCR5 mAb and binding kinetics sensorgram is recorded.

FIG. 2 shows a Biacore kinetics assay sensorgram of 3 mAb clones (16D7 in FIG. 2A; 79E7 in FIG. 2B; and MAB190 in FIG. 2C) against hCXCR5 by the conventional peptide-based assay format shown in FIG. 1.

FIG. 3 shows the Kₒₙ/K_{off} rate map of 3 mAb clones against hCXCR5 by the conventional peptide-based assay format. The results demonstrate good reproducibility; the three mAbs exhibited similar affinity (K_{D}; see also Table 1).

FIG. 4 is a schematic diagram showing the reverse-format peptide-based assay for antibodies against GPCR. In this format, the same hCXCR5 N-terminal peptide as shown in FIG. 1 was biotinylated and captured on a streptavidin (SA) SPR chip. Anti-hCXCR5 mAbs were then injected over the captured peptide.

FIG. 5 shows Biacore kinetics assay sensorgrams of 3 mAb clones (16D7 in FIG. 5A; 79E7 in FIG. 5B; and MAB190 in FIG. 5C) against hCXCR5 using the reversed-format peptide-based assay.

FIG. 6 shows the Kₒₙ/K_{off} rate map of 3 mAb clones against hCXCR5 by the reversed-format peptide-based assay. In this assay format, clone 16D7 showed more then 10-fold slower off rate, which was not detected using the conventional assay as shown in FIG. 2.

FIG. 7 shows Biacore sensorgrams showing the curve shape impacted by different levels of peptides used in the reversed-format peptide-based assay.

FIG. 8 is the Kₒₙ/K_{off} rate map for different antibodies and various levels of peptides used in the reversed-format peptide-based assay: 16D7 and 5 RU in FIG. 8A; 16D7 and 20 RU in FIG. 8B; 16D7 and 100 RU in FIG. 8C; MAB190 and 5 RU in FIG. 8D; MAB190 and 20 RU in FIG. 8E; and MAB190 and 100 RU in FIG. 8F. The results of experiments repeated in triplicate are shown. Higher levels of peptides used in the assay had a significant impact on the slow off-rate of antibody 16D7. When the captured peptide level was low (<20 RU), this assay format was reproducible and reliable.

FIG. 9 shows an SDS-PAGE gel (FIG. 9A) and Western blot (FIG. 9B) analysis of hCXCR5 protein purified via C-terminal 6×His tag after expression using a baculovirus expression system.

FIG. 10 shows Biacore sensorgrams for hCXCR5 whole-receptor surface preparation using different concentrations of NHS/EDC as the stabilization/crosslinking agent. Same levels (500RUs) of hCXCR5 were captured on Fc2 (flow cell 2), Fc3, and Fc4, respectively. Fc2 was injected with the running buffer HBS—N for 7 min, Fc3 was injected with 20 μM NHS and 5 μM EDC for 7 min, and Fc4 was injected with 200 μM NHS and 50 μM EDC for 7 min. All four flow cells were inactivated by injection of 1M ethanolamine for 7 min.

FIG. 11 shows Biacore sensorgrams generated with different levels of NHS/EDC as the stabilization/crosslinking agent.

FIG. 12 shows Biacore sensorgrams for different mAb clones (16D7 in FIG. 12A and FIG. 12D; MAB190 in FIG. 12B and FIG. 12E; and CXCL13 in FIG. 12C and FIG. 12F) generated with different crosslinkers as the stabilization/crosslinking agent on a NTA chip surface.

FIG. 13 shows Biacore sensorgrams for different mAb clones (16D7 in FIG. 13A and FIG. 13D; MAB190 in FIG. 13B and FIG. 13E; and CXCL13 in FIG. 13C and FIG. 13F) generated with different crosslinkers as the stabilization/crosslinking agent on a CM5 chip surface.

FIG. 14 shows Biacore kinetics assay sensorgrams for different mAb clones generated using different levels of hCXCR5 receptor (16D7 and 300 RU in FIG. 14A; 79E7 and 300 RU in FIG. 14B; CXCL13 and 300 RU in FIG. 14C; MAB190 and 300 RU in FIG. 14D; 16D7 and 700 RU in FIG. 14E; 79E7 and 700 RU in FIG. 14F; CXCL13 and 700 RU in FIG. 14G; MAB190 and 700 RU in FIG. 14H) captured and stabilized on a NTA chip surface.

FIG. 15 shows Kₒₙ/K_{off} rate maps generated using different levels of hCXCR5 receptor captured and stabilized on NTA chip surface. This assay format was able to differentiate between three mAb clones. In addition, the whole receptor assay enabled direct comparison of antibodies with their ligands.

FIG. 16 shows SDS-PAGE gel (FIG. 16A) and Western blot (FIG. 16B) analyses of CD52 VLP samples produced by HEK293FT cell transient expression. Lanes: 1. Sf9 cell negative control membrane prep, 2. CD52 positive control protein produced in Sf9 cell, 3. HEK293FT negative control total cell lysates, 4. HEK293FT total cell lysate transiently expressing CD52, 5. negative control VLP prep, 6. CD52 VLP prep from HEK293FT cells.

FIG. 17 shows Biacore sensorgrams for capturing and stabilization of CD52 VLP on a C1 chip surface. FIG. 17A: immobilization of anti-CD52 (Campath) to C1 chip surface; FIG. 17B: capturing and stabilization of CD52 VLP on C1 chip surface via anti-CD52 mAb.

FIG. 18 shows Biacore kinetics assay sensorgrams of anti-CD52 mAbs generated using CD52 VLP captured and stabilized on a C1 chip surface.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “protein” means one or more proteins.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The present disclosure provides methods of stabilizing captured membrane proteins, for example, membrane receptors such as G-protein-coupled receptors (GPCRs), on analytical surfaces, for example, surface plasmon resonance surfaces such as Biacore sensor chip surfaces, by limited chemical crosslinking. In some embodiments, the methods of the invention comprise purified or pre-enriched native GPCR protein immobilized on Biacore sensor chip surfaces via different capturing agents and further stabilized by a variety of crosslinkers. The resultant stabilized receptor proteins retain good ligand-binding activity and can be accessed by receptor specific antibodies. This limited chemical crosslinking is efficient and reliable. It enables high quality kinetics assays of membrane proteins via the same methods that are conventionally used for soluble proteins. Also described here are methods for capturing and stabilizing membrane protein complexes on analytical surfaces for binding kinetics assays by limited chemical crosslinking. In some embodiments, the membrane protein complexes are virus like particles (VLPs) with specific cell surface receptors displayed on the VLP surface.

As used herein, the terms “polypeptide,” “protein,” and “peptide” are interchangeable and refer to a chain of amino acid monomers linked by peptide bonds. Typically, polypeptide chains are unbranched. As used herein, the terms “residue” and “protein residue” are interchangeable and refer to an amino acid that is bonded with other amino acids by one or more peptide bonds within a protein.

As used herein, the term “membrane” refers to a phospholipid bilayer that surrounds a cell or organelle. For example, a cytoplasmic membrane is a phospholipid bilayer that surrounds a cell, whereas a nuclear membrane is a phospholipid membrane that surrounds a nucleus. Other organelles that comprise phospholipid bilayer membranes include mitochondria, chloroplasts, the Golgi apparatus, and the endoplasmic reticulum.

As used herein, the terms “membrane protein” or “membrane-associated protein” are interchangeable and refer to a protein attached to, or associated with, the membrane of a cell or organelle. Membrane proteins typically are specifically targeted to different types of biological membranes. They are also the target of over 50% of all modern medicinal drugs. It is estimated that 20-30% of all genes in most genomes encode membrane proteins. Classes of membrane proteins include integral membrane proteins, peripheral membrane proteins, and membrane-associated peptides.

Integral membrane proteins are permanently attached to the membrane. Such proteins can be separated from the biological membranes using detergents, nonpolar solvents, and sometimes denaturing agents. They can be classified according to their relationship with the bilayer. Integral polytopic proteins, also known as “transmembrane proteins,” are integral membrane proteins, which span across the membrane at least once. They have one of two tertiary structures: helix bundle proteins which are present in all types of biological membranes; and beta barrel proteins which are found only in outer membranes of Gram-negative bacteria, lipid-rich cell walls of a few Gram-positive bacteria, and outer membranes of mitochondria and chloroplasts. Integral monotopic proteins are integral membrane proteins which are attached to only one side of the membrane and do not span the whole way across.

Peripheral membrane proteins are temporarily attached either to the lipid bilayer or to integral proteins by one or a combination of hydrophobic, electrostatic, and/or other non-covalent interactions. Peripheral membrane proteins can be made to dissociate from the membrane by treatment with a polar reagent, such as a solution with an elevated pH or high salt concentration.

Membrane-associated peptides include polypeptide toxins and many antibacterial peptides, such as colicins or hemolysins, and certain proteins involved in apoptosis. These proteins are water-soluble but can aggregate and associate irreversibly with the lipid bilayer and become reversibly or irreversibly membrane-associated. For example, the antibacterial peptide magainin-2, from Xenopus laevis, is an amphipathic peptide that associates with lipid bilayers and disrupts membrane structure after insertion.

As used herein, the terms “membrane protein complex” and “membrane-associated protein complex” are interchangeable and can refer to a complex of membrane proteins, optionally comprising non-protein components, such as phospholipids. Alternatively, a membrane protein complex can be a complex of more than one protein that is associated with or attached to the membrane of a cell or organelle.

One non-limiting example of a membrane protein complex is a virus-like particle (VLP), which is a particle that resembles a virus, but is non-infectious because it does not contain any viral genetic material. The expression of viral structural proteins, such as envelope or capsid, can result in the self-assembly of virus like particles (VLPs). VLPs derived from the Hepatitis B virus and composed of the small HBV derived surface antigen (HBsAg) were described over 40 years ago from patient sera. More recently, VLPs have been produced from components of a wide variety of virus families including Parvoviridae (e.g. adeno-associated virus), Retroviridae (e.g. HIV), and Flaviviridae (e.g. Hepatitis C virus). VLPs can be produced in a variety of cell culture systems including mammalian cell lines, insect cell lines, yeast, and plant cells.

A sub-type of VLPs are lipoparticles, which are VLPs developed to aid the study of integral membrane proteins. Lipoparticles are stable, highly purified, homogeneous VLPs that are engineered to contain high concentrations of one or more conformationally intact membrane proteins of interest. Because of their hydrophobic domains, integral membrane proteins are insoluble in aqueous solution (at least in their native conformation) and are thus difficult to manipulate outside of living cells. Lipoparticles can incorporate a wide variety of structurally intact membrane proteins, including G protein-coupled receptors (GPCRs), ion channels, and viral envelopes. Lipoparticles provide a platform for numerous applications including antibody screening, production of immunogens, and ligand binding assays. Some lipoparticles comprise a protein core surrounded by a phospholipid bilayer that comprises membrane proteins.

As used herein, “primary structure” refers to the sequence of a biopolymer, such as the amino acid sequence of a protein or peptide.

As used herein, “secondary structure” refers to the general three-dimensional form of local segments of biopolymers such as proteins and nucleic acids (DNA/RNA). For example, in a protein, secondary structure comprises both alpha helices and beta sheets. Secondary structure can be formally defined by the hydrogen bonds of the biopolymer, as observed in an atomic-resolution structure. In proteins, the secondary structure is defined by the patterns of hydrogen bonds between backbone amino and carboxyl groups. In nucleic acids, the secondary structure is defined by the hydrogen bonding between the nitrogenous bases.

As used herein, “tertiary structure” refers to the three-dimensional structure of a protein or other macromolecule, as defined by its atomic coordinates. Proteins and nucleic acids are capable of diverse functions ranging from molecular recognition to catalysis. Such functions require a precise three-dimensional tertiary structure. Thus, in many cases, loss of tertiary structure is associated with a loss of functionality. For membrane proteins, such as integral membrane proteins, removal from the membrane environment is often associated with a loss of tertiary structure because membrane proteins contain many hydrophobic domains, which mis-fold when moved to a hydrophilic environment.

As used herein, “quaternary structure” refers to the arrangement of multiple folded protein or coiling protein molecules in a multi-subunit complex. For nucleic acids, the term is less common, but can refer to the higher-level organization of DNA in chromatin, including its interactions with histones, or to the interactions between separate RNA units in the ribosome or spliceosome.

As used herein, the term “analytical surface” refers to a surface used for chemical, physical, biophysical, biochemical, and/or biological analysis. Non-limiting examples of analytical surfaces include surfaces used for optical microscopy, atomic force microscopy, surface plasmon resonance analysis, Raman spectroscopy, fluorometry, fluorescence microscopy, electron microscopy, x-ray spectroscopy, and any other technique used for analyzing molecules or other analytes at a surface.

As used herein, the term “immobilize” refers to the capture or attachment of a molecule, such as a protein, on an insoluble material, such as an analytical surface.

As used herein, the term “surface plasmon resonance” (“SPR”) can refer both to a physical phenomenon and the analytical technique that employs the physical phenomenon. SPR is the collective oscillation of electrons in a solid or liquid stimulated by incident light. The resonance condition is established when the frequency of light photons matches the natural frequency of surface electrons oscillating against the restoring force of positive nuclei. SPR in nanometer-sized structures is called localized surface plasmon resonance. SPR is the basis of many standard tools for measuring adsorption of material onto planar metal (typically gold and silver) surfaces or onto the surface of metal nanoparticles. It is the fundamental principle behind many color-based biosensor applications and different lab-on-a-chip sensors.

Biacore pioneered commercial SPR biosensors offering a unique technology for collecting high-quality, information-rich data from biomolecular binding events. Since the release of the first instrument in 1990, researchers around the world have used Biacore's optical biosensors to characterize binding events with samples ranging from proteins, nucleic acids, small molecules to complex mixtures, lipid vesicles, viruses, bacteria, and eukaryotic cells. Other SPR systems include Bio-Rad ProteOn XPR36, Eco Chemie Autolab TWINGLE, Reichert Inc. SR7000DC, and Sierra Sensors GmbH.

SPR reflectivity measurements can be used to detect molecular adsorption, such as that of polymers, DNA, or proteins. In some cases, the angle of the reflection minimum (i.e. the absorption maximum) is measured. This angle changes on the order of 0.1° during thin (about nm thickness) film adsorption. In other cases the changes in the absorption wavelength is measured. The mechanism of detection is based on the fact that the adsorbing molecules cause changes in the local index of refraction, changing the resonance conditions of the surface plasmon waves.

When the affinity of two ligands is to be determined, the binding constant K_{D} can be determined as K_{D}=k_{d}/kₐ, where k_{d} is the dissociation rate constant and kₐ is the association rate constant. This value can also be found using dynamical SPR parameters. For this, a “bait” molecule is immobilized on an SPR surface. Through a microflow system, a solution with the molecule's binding partner (the “prey” molecule) is injected over the immobilized bait layer. As the prey analyte binds the bait ligand, an increase in SPR signal (expressed in response units, RU) is observed. After desired association time, a solution without the prey analyte (usually the buffer) is injected over the surface that dissociates the bound complex between bait ligand and prey analyte. As the prey analyte dissociates from the bait ligand, a decrease in SPR signal (expressed in resonance units, RU) is observed. From these association (“on rate,” kₐ or kₒₙ) and dissociation (“off rate,” k_{d} or k_{off}) rates, the equilibrium dissociation constant (“binding constant,” K_{D}) can be determined. The actual SPR signal can be explained by the electromagnetic “coupling” of the incident light with the surface plasmon of the gold layer. This plasmon can be influenced by the layer just a few nanometers across the gold-solution interface, i.e. the bait protein and possibly the prey protein. Binding makes the reflection angle change.

Biacore SPR sensor chips are available with a variety of types of surfaces. For example, CM3, CM4, CM5, and CM7 chips comprise dextran chains that are covalently bonded to the chip surface, and which contain carboxyl groups at their free ends. These chips can be used to attach proteins, nucleic acids, carbohydrates or small molecules to the chip surface by coupling the molecules to the carboxyl groups on the sensor surface via —NH2, —SH, —CHO, —OH or —COOH moieties. C1 chips comprise a matrix-free surface (i.e. no dextran chains) that is carboxymethylated; like the CM chips, the surface comprises free carboxyl groups that can be used to attach molecules via —NH2, —SH, —CHO, —OH or —COOH moieties. Streptavidin (SA) chips comprise carboxymethylated dextran pre-immobilized with streptavidin for immobilization of biotinylated interaction partners and is capable of high-affinity capture of biotinylated ligands such as proteins, peptides, nucleic acids or carbohydrates. NTA chips comprise carboxymethylated dextran pre-immobilized with nitrilotriacetic acid (NTA). His-tagged molecules are immobilized via Ni²⁺/NTA chelation. These, and other sensor chips and analytical surfaces may be used with the methods and apparatuses disclosed herein.

As used herein, the term “capture moiety” refers to a chemical moiety attached to a molecule that can be used to capture or immobilize the molecule, for example, through interaction with another chemical moiety. For example, a poly-histidine tag (His-tag, 6×His-tag, hexa histidine-tag, or His6-tag) is a capture moiety comprising at least six histidine amino acid residues that can be used to capture or immobilize a His-tagged molecule because the string of histidine residues binds to several types of immobilized metal ions, including nickel, cobalt and copper, under specific buffer conditions. In addition, anti-His-tag antibodies are commercially available for use in assay methods involving His-tagged proteins. Any protein for which an antibody specific for that protein exists can comprise a capture moiety. For example, CD52 can be a capture moiety, which is captured using an anti-CD52 antibody. CD81 can be a capture moiety, which is captured using an anti-CD81 antibody.

As used herein, the term “crosslink” or “cross-link” refers to a bond, such as a covalent bond or ionic bond, that links one polymer chain, such as a biopolymer or synthetic polymer, to another.

As used herein, the terms “thiol,” “thiol moiety,” “thiol group,” “sulfhydryl,” “sulfhydryl group,” and “sulfhydryl moiety” are interchangeable and refer to a —SH functional group in a molecule.

As used herein, the terms “amine,” “amine group,” and “amine moiety” are interchangeable and refer to a —NH functional group in a molecule.

As used herein, the terms “carboxyl group,” “carboxyl moiety,” “carboxy group,” and “carboxy moiety” are interchangeable and refer to a functional group consisting of a carbonyl (RR′C═O) and a hydroxyl (R—O—H), which has the formula —C(═O)OH, also written as —COOH or —CO₂H.

As used herein, the terms “carbohydrate group,” “carbohydrate moiety,” “saccharide group,” or “saccharide moiety” are interchangeable and refer to carbohydrate or sugar groups, such as monosaccharide, disaccharide, oligosaccharide, or polysaccharide groups, that are covalently attached to a molecule.

As used herein, the terms “crosslinking reagent,” “crosslinking agent,” or “crosslinker” are interchangeable and refer to a reagent or set of reagents capable of chemically linking two molecules, for example two proteins, by one or more covalent bonds. Crosslinking reagents contain two or more reactive ends that are capable of attaching to specific functional groups (primary amines, sulfhydryls, etc.) on proteins or other molecules, and can be broadly grouped into homobifunctional and heterobifunctional crosslinkers.

Homobifunctional crosslinking reagents have the same type of reactive group at either end of the crosslinker and thus link two functional groups of the same type, forming, for example, amine-to-amine or thiol-to-thiol linkages. Examples of crosslinkers that form amine-to-amine linkages include, but are not limited to:
disuccinimidyl glutarate (DSG);
disuccinimidyl suberate (DSS);
disuccinimidyl tartarate (DST);
bis(sulfosuccinimidyl)suberate (BS3);
tris-succinimidyl aminotriacetate (TSAT);
bis(succinimidyl)penta(ethylene glycol) (BS(PEG)₅);
bis(succinimidyl) nona(ethylene glycol) (BS(PEG)₉);
dithiobis(succinimidyl propionate) (DSP);
3,3″-dithiobis(sulfosuccinimidylpropionate) (DTSSP);
bis[2-(succinimidooxycarbonyloxy)ethyl]sulfone (BSOCOES);
ethylene glycol bis(succinimidylsuccinate) (EGS);
ethylene glycol bis(sulfosuccinimidylsuccinate) (sulfo-EGS);
dimethyl adipimidate.2HCl (DMA);
dimethyl pimelimidate.2HCl (DMP);
dimethyl suberimidate.2HCl (DMS);
dimethyl 3,3″-dithiobispropionimidate.2HCl (DTBP); and
1,5-Difluoro-2,4-dinitrobenzene (DFDNB).

Examples of homobifunctional crosslinkers that form thiol-to-thiol linkages include, but are not limited to:
bismaleimidoethane (BMOE);
1,4-bismaleimidobutane (BMB);
bismaleimidohexane (BMH);
tris(2-maleimidoethyl)amine (TMEA), a tri-functional crosslinker capable of three-way crosslinking between three thiol moieties;
1,8-bismaleimido-diethyleneglycol (BM(PEG)₂);
1,11-bismaleimido-triethyleneglycol (BM(PEG)₃);
1,4-bismaleimidyl-2,3-dihydroxybutane (BMDB); and
dithiobismaleimidoethane (DTME).

Heterobifunctional crosslinking reagents have different types of reactive groups at either end of the crosslinker and thus link two functional groups of different types, forming, for example, amine-to-thiol or carboxyl-to-amine, thiol-to-carbohydrate, or hydroxyl-to-thiol linkages. Examples of heterobifunctional crosslinking reagents that form amine-to-thiol (amine-to-sulfhydryl) linkages include, but are not limited to:
N-α-maleimidoacetoxy succinimide ester (AMAS);
N-β-maleimidopropyloxy succinimide ester (BMPS);
N-γ-maleimidobutyryloxy succinimide ester (GMBS);
N-γ-maleimidobutyryloxy sulfosuccinimide ester (sulfo-GMBS);
N-ε-maleimidocaproyloxy succinimide ester (EMCS);
N-ε-maleimidocaproyloxy sulfosuccinimide ester (sulfo-EMCS);
N-κ-maleimidoundecanoyloxy succinimide ester (KMUS);
N-κ-maleimidoundecanoyloxy sulfosuccinimide ester (sulfo-KMUS);
m-maleimidobenzoyl-N-hydroxysuccinimide ester (MBS);
m-maleimidobenzoyl-N-hydroxysulfosuccinimide ester (sulfo-MBS);
succinimidyl 4-(N-maleimidomethyl)cyclohexane-1-carboxylate (SMCC);
sulfosuccinimidyl 4-(N-maleimidomethyl)cyclohexane-1-carboxylate (sulfo-SMCC);
succinimidyl 4-(p-maleimidophenyl)butyrate (SMPB);
sulfosuccinimidyl 4-(p-maleimidophenyl)butyrate (sulfo-SMPB);
succinimidyl 6[(β-maleimidopropionamido)hexanoate](SMPH);
succinimidyl 4-(N-maleimidomethyl)cyclohexane-1-carboxy-(6-amidocaproate) (long-chain SMCC or LC-SMCC);
polyethylene glycol functionalized with succinimidyl and maleimido ends with n ethylene glycol units (SM(PEG)ₙ), for example, SM(PEG)₂, SM(PEG)₄, SM(PEG)₆, SM(PEG)₈, SM(PEG)₁₂, and SM(PEG)₂₄;
succinimidyl 3-(2-pyridyldithio)propionate (SPDP);
succinimidyl 6-[3(2-pyridyldithio) propionamido]hexanoate (long-chain SPDP or LC-SPDP);
2-pyridyldithiol-tetraoxatetradecane-N-hydroxysuccinimide (PEG4-SPDP);
2-pyridyldithiol-tetraoxaoctatriacontane-N-hydroxysuccinimide (PEG12-SPDP);
4-succinimidyloxycarbonyl-alpha-methyl-alpha(2-pyridyldithio)toluene (SMPT);
succinimidyl iodoacetate (SIA);
succinimidyl 3-(bromoacetamido)propionate (SBAP);
succinimidyl (4-iodoacetyl)aminobenzoate (STAB); and
sulfosuccinimidyl (4-iodoacetyl)aminobenzoate (sulfo-SIAB).

Examples of heterobifunctional crosslinking reagents that form carboxyl-to-amine linkages include, but are not limited to:
dicyclohexylcarbodiimide (DCC) with N-hydroxysuccinimide (NHS) or N-hydroxysulfosuccinimide (NHSS or sulfo-NHS); and
1-ethyl-3-(3-dimethylaminopropyl) carbodiimide (EDC) with N-hy dr oxy succinimide (NHS) or N-hydroxysulfosuccinimide (NHSS or sulfo-NHS).

An example of a heterobifunctional crosslinking reagent that forms thiol-to-hydroxyl linkages is p-maleimidophenyl isocyanate (PMPI).

Examples of heterobifunctional crosslinking reagents that form thiol-to-carbohydrate linkages include, but are not limited to:
N-β-maleimidopropionic acid hydrazide-TFA (BMPH);
N-ε-maleimidocaproic acid hydrazide-TFA (EMCH);
N-κ-maleimidoundecanoic acid hydrazide-TFA;
4-(4-N-maleimidophenyl)butyric acid hydrazide-HCl (MPBH); and
3-(2-pyridyldithio)propionyl hydrazide (PDPH).

In some embodiments of the methods and apparatuses disclosed herein, the amount of crosslinking is limited so as not to interfere with the activity of the crosslinked protein or protein complex. In some embodiments, the amount of crosslinking is more than a lower threshold, such that the protein or protein complex is stabilized, but is less than an upper threshold, at which the activity is diminished or modulated or altered to an unacceptable degree.

In some embodiments, the amount of crosslinking is assessed according to the average number of crosslinks per protein or protein complex. For example, a desired number of crosslinks per immobilized protein or protein complex according to the methods and apparatuses disclosed herein could be about 1, or about 2, or about 3, or about 4, or about 5, or about 6, or about 7, or about 8, or about 9, or about 10, or about 15, or about 20, or about 30 crosslinks per protein or protein complex.

In some embodiments, the amount of crosslinking is assessed according to the function of the immobilized protein or protein complex, and more specifically according to the affinity or ability of the protein or protein complex to bind to one or more of its binding partners. As used herein, the term “binding partner” refers to any molecule that a membrane protein or complex is capable of binding or interacting with, for example, but not limited to, one or more of the protein or protein complex's ligands, receptors, epitopes, substrates, co-factors, co-complexes, sub-domains, units, subunits, etc. In some embodiments, the amount of crosslinking preserves about 40%, or about 50%, or about 60%, or about 70%, or about 80%, or about 90%, or about 100% of the binding activity of the protein or protein complex for its binding partner. In other embodiments, the amount of crosslinking decreases the binding activity of the protein or protein complex for its binding partner only by about 1%, or about 2%, or about 5%, or about 10%, or about 15%, or about 20%, or about 30%, or about 40%, or about 50%.

In some embodiments, the amount of crosslinking is assessed or determined according to the amount or concentration of the crosslinking reagent that is used. This in turn is dependent on the particular crosslinking reagent employed. As a non-limiting example, NHS/EDC may be used in a ratio of 4:1 at about 4 nM NHS and 1 nM EDC, or about 20 nM NHS and 5 nM EDC, or about 200 nM NHS and 50 nM EDC, or about 4 μM NHS and 1 μM EDC, or about 20 μM NHS and 5 μM EDC, or about 200 μM NHS and 50 μM EDC. Thus, EDC may be used at concentrations from about 1 nM to about 1 mM, and NHS may also be used at concentrations from about 1 nM to about 1 mM. In the methods and apparatuses described herein, the optimal concentrations of crosslinking reagents to be used will adequately stabilize an immobilized protein without resulting in an unacceptable decrease or loss in protein functionality or binding affinity.

In one aspect, the invention provides methods for immobilizing a membrane protein or membrane protein complex on an analytical surface, the methods comprising: (a) obtaining a membrane protein or membrane protein complex comprising a capture moiety; (b) immobilizing the membrane protein or membrane protein complex on the analytical surface by means of the capture moiety; and (c) stabilizing at least one of the secondary, tertiary, or quaternary structures of the immobilized membrane protein or membrane protein complex by crosslinking the immobilized membrane protein or membrane protein complex with a crosslinking reagent.

In some embodiments of this aspect, the analytical surface is a surface plasmon resonance (SPR) surface. In some embodiments, the capture moiety is a 6×His tag and the analytical surface is a nickel-nitrilotriacetic acid (Ni-NTA) surface. In some embodiments, the crosslinking reagent forms a linkage between an amine moiety and a carboxylic acid moiety, and in certain embodiments, the crosslinking reagent comprises: (i) N-hydroxysulfosuccinimide (NHSS) and 1-ethyl-3-(3-dimethylaminopropyl)carbodiimide (EDC); or (ii) N-hydroxysuccinimide (NHS) and 1-ethyl-3-(3-dimethylaminopropyl)carbodiimide (EDC). In some embodiments, the crosslinking reagent forms a linkage between a thiol moiety and an amine moiety, and in certain embodiments, the crosslinking reagent comprises: (i) succinimidyl 4-(N-maleimidomethyl)cyclohexane-1-carboxylate (SMCC); or (ii) 3-(2-pyridyldithio)propionate (SPDP). In some embodiment, the crosslinking reagent forms a linkage between two amine moieties, and in certain embodiments, the crosslinking reagent comprises bis(succinimidyl)penta(ethylene glycol) (BS(PEG)₅). In some embodiments, the method of claim 1, wherein the crosslinking reagent forms a linkage between two thiol moieties, and in certain embodiments, the crosslinking reagent comprises: (i) dithiobismaleimidoethane (DTME); or (ii) 1,8-bismaleimido-diethyleneglycol (BM(PEG)₂). In some embodiments, the crosslinking reagent forms a linkage between two carbohydrate moieties, and in certain embodiments, crosslinking reagent comprises: (i) S-(2-thiopyridyl)-L-cysteine hydrazide (TPCH); or (ii) S-(2-thiopyridyl)mercapto-propionohydrazide (TPMPH). In some embodiments, the crosslinking reagent forms a linkage between a carbohydrate moiety and a thiol moiety, and in certain embodiments, the crosslinking reagent comprises: (i) N-beta-maleimidopropionic acid hydrazide (BMPH); or (ii) 3-(2-pyridyldithio)propionyl hydrazide (PDPH). In some embodiments, the membrane protein or membrane protein complex is capable of binding with a binding partner after crosslinking.

In another aspect, the invention provides methods for immobilizing a membrane protein or membrane protein complex on an analytical surface, the methods comprising: (a) obtaining a membrane protein or membrane protein complex modified with a capture moiety; (b) coupling to the analytical surface an antibody specific for the capture moiety using a coupling reagent; (c) immobilizing the membrane protein or membrane protein complex on the analytical surface by means of interaction between the antibody and the capture moiety; and (d) stabilizing at least one of the secondary, tertiary, or quaternary structures of the immobilized membrane protein or membrane protein complex by crosslinking the immobilized membrane protein or membrane protein complex with a crosslinking reagent.

In some embodiments of this aspect, the analytical surface is a surface plasmon resonance (SPR) surface. In some embodiments, the capture moiety is a 6×His tag and the antibody is an anti-6×His antibody. In some embodiments, the membrane protein complex is a virus-like particle (VLP). In some embodiments, the capture moiety is a protein or protein complex present on the surface of the VLP and the antibody is specific to the capture moiety. In some embodiments, the capture moiety is CD52 and the antibody is an anti-CD52 antibody. In some embodiments, the capture moiety is CD81 and the antibody is an anti-CD81 antibody. In some embodiments, the analytical surface is a surface comprising free carboxyl groups and the coupling reagent comprises N-hydroxysulfosuccinimide (NHS) and 1-ethyl-3-(3-dimethylaminopropyl)carbodiimide (EDC). In some embodiments, the surface comprising free carboxyl groups is a CM5 sensor chip surface or a C1 sensor chip surface.

In some embodiments of this aspect, the crosslinking reagent forms a linkage between an amine moiety and a carboxylic acid moiety, and in certain embodiments, the crosslinking reagent comprises: (i) N-hydroxysulfosuccinimide (NHSS) and 1-ethyl-3-(3-dimethylaminopropyl)carbodiimide (EDC); or (ii) N-hydroxysuccinimide (NHS) and 1-ethyl-3-(3-dimethylaminopropyl)carbodiimide (EDC). In some embodiments, the crosslinking reagent forms a linkage between a thiol moiety and an amine moiety, and in certain embodiments, the crosslinking reagent comprises: (i) succinimidyl 4-(N-maleimidomethyl)cyclohexane-1-carboxylate (SMCC); or (ii) 3-(2-pyridyldithio)propionate (SPDP). In some embodiment, the crosslinking reagent forms a linkage between two amine moieties, and in certain embodiments, the crosslinking reagent comprises bis(succinimidyl)penta(ethylene glycol) (BS(PEG)₅). In some embodiments, the method of claim 1, wherein the crosslinking reagent forms a linkage between two thiol moieties, and in certain embodiments, the crosslinking reagent comprises: (i) dithiobismaleimidoethane (DTME); or (ii) 1,8-bismaleimido-diethyleneglycol (BM(PEG)₂). In some embodiments, the crosslinking reagent forms a linkage between two carbohydrate moieties, and in certain embodiments, crosslinking reagent comprises: (i) S-(2-thiopyridyl)-L-cysteine hydrazide (TPCH); or (ii) S-(2-thiopyridyl)mercapto-propionohydrazide (TPMPH). In some embodiments, the crosslinking reagent forms a linkage between a carbohydrate moiety and a thiol moiety, and in certain embodiments, the crosslinking reagent comprises: (i) N-beta-maleimidopropionic acid hydrazide (BMPH); or (ii) 3-(2-pyridyldithio)propionyl hydrazide (PDPH). In some embodiments, the membrane protein or membrane protein complex is capable of binding with a binding partner after crosslinking.

In another aspect, the invention provides analytical surfaces comprising: a membrane protein or membrane protein complex comprising a capture moiety, wherein the membrane protein or membrane protein complex is immobilized on the analytical surface by means of the capture moiety, and wherein at least one of the secondary, tertiary, or quaternary structures of the membrane protein or membrane protein complex is stabilized by crosslinking. In some embodiments of this aspect, the analytical surface is a surface plasmon resonance (SPR) surface. In some embodiments, the capture moiety is a 6×His tag and the analytical surface is a nickel-nitrilotriacetic acid (Ni-NTA) surface.

In some embodiments of this aspect, the membrane protein or membrane protein complex is crosslinked using a crosslinking reagent that forms a linkage between an amine moiety and a carboxylic acid moiety, and in certain embodiments, the crosslinking reagent comprises: (i) N-hydroxysulfosuccinimide (NHSS) and 1-ethyl-3-(3-dimethylaminopropyl)carbodiimide (EDC); or (ii) N-hydroxysuccinimide (NHS) and 1-ethyl-3-(3-dimethylaminopropyl)carbodiimide (EDC). In some embodiments, the membrane protein or membrane protein complex is crosslinked using a crosslinking reagent that forms a linkage between a thiol moiety and an amine moiety, and in certain embodiments, the crosslinking reagent comprises: (i) succinimidyl 4-(N-maleimidomethyl)cyclohexane-1-carboxylate (SMCC); or (ii) 3-(2-pyridyldithio)propionate (SPDP). In some embodiments, the membrane protein or membrane protein complex is crosslinked using a crosslinking reagent that forms a linkage between two amine moieties, and in certain embodiments, the crosslinking reagent comprises bis(succinimidyl)penta(ethylene glycol) (BS(PEG)₅). In some embodiments, the membrane protein or membrane protein complex is crosslinked using a crosslinking reagent that forms a linkage between two thiol moieties, and in certain embodiments, the crosslinking reagent comprises: (i) dithiobismaleimidoethane (DTME); or (ii) 1,8-bismaleimido-diethyleneglycol (BM(PEG)₂). In some embodiments, the membrane protein or membrane protein complex is crosslinked using a crosslinking reagent that forms a linkage between two carbohydrate moieties, and in certain embodiments, the crosslinking reagent comprises: (i) S-(2-thiopyridyl)-L-cysteine hydrazide (TPCH); or (ii) S-(2-thiopyridyl)mercapto-propionohydrazide (TPMPH). In some embodiments, the membrane protein or membrane protein complex is crosslinked using a crosslinking reagent that forms a linkage between a carbohydrate moiety and a thiol moiety, and in certain embodiments, the crosslinking reagent comprises: (i) N-beta-maleimidopropionic acid hydrazide (BMPH); or (ii) 3-(2-pyridyldithio)propionyl hydrazide (PDPH). In some embodiments, the membrane protein or membrane protein complex is capable of binding with a binding partner after crosslinking.

In another aspect, the invention provides analytical surfaces comprising: (a) a membrane protein or membrane protein complex comprising a capture moiety, and (b) an antibody specific for the capture moiety, wherein the antibody is coupled to the analytical surface using a coupling reagent, wherein the membrane protein or membrane protein complex is immobilized on the analytical surface by means of interaction between the antibody and the capture moiety, and wherein at least one of the secondary, tertiary, or quaternary structures of the membrane protein or membrane protein complex is stabilized by crosslinking. In some embodiments of this aspect, the analytical surface is a surface plasmon resonance (SPR) surface. In some embodiments, the capture moiety is a 6×His tag and the antibody is an anti-6×His antibody. In some embodiments, the membrane protein complex is a virus-like particle (VLP). In some embodiments, the capture moiety is a protein or protein complex present on the surface of the VLP and the antibody is specific to the capture moiety. In some embodiments, the capture moiety is CD52 and the antibody is an anti-CD52 antibody. In some embodiments, the capture moiety is CD81 and the antibody is an anti-CD81 antibody. In some embodiments, the analytical surface is a surface comprising free carboxyl groups and the coupling reagent comprises N-hydroxysulfosuccinimide (NHS) and 1-ethyl-3-(3-dimethylaminopropyl)carbodiimide (EDC). In some embodiments, the surface comprising free carboxyl groups is a CM5 sensor chip surface or a C1 sensor chip surface.

In some embodiments of this aspect, the membrane protein or membrane protein complex is crosslinked using a crosslinking reagent that forms a linkage between an amine moiety and a carboxylic acid moiety, and in certain embodiments, the crosslinking reagent comprises: (i) N-hydroxysulfosuccinimide (NHSS) and 1-ethyl-3-(3-dimethylaminopropyl)carbodiimide (EDC); or (ii) N-hydroxysuccinimide (NHS) and 1-ethyl-3-(3-dimethylaminopropyl)carbodiimide (EDC). In some embodiments, the membrane protein or membrane protein complex is crosslinked using a crosslinking reagent that forms a linkage between a thiol moiety and an amine moiety, and in certain embodiments, the crosslinking reagent comprises: (i) succinimidyl 4-(N-maleimidomethyl)cyclohexane-1-carboxylate (SMCC); or (ii) 3-(2-pyridyldithio)propionate (SPDP). In some embodiments, the membrane protein or membrane protein complex is crosslinked using a crosslinking reagent that forms a linkage between two amine moieties, and in certain embodiments, the crosslinking reagent comprises bis(succinimidyl)penta(ethylene glycol) (BS(PEG)₅). In some embodiments, the membrane protein or membrane protein complex is crosslinked using a crosslinking reagent that forms a linkage between two thiol moieties, and in certain embodiments, the crosslinking reagent comprises: (i) dithiobismaleimidoethane (DTME); or (ii) 1,8-bismaleimido-diethyleneglycol (BM(PEG)₂). In some embodiments, the membrane protein or membrane protein complex is crosslinked using a crosslinking reagent that forms a linkage between two carbohydrate moieties, and in certain embodiments, the crosslinking reagent comprises: (i) S-(2-thiopyridyl)-L-cysteine hydrazide (TPCH); or (ii) S-(2-thiopyridyl)mercapto-propionohydrazide (TPMPH). In some embodiments, the membrane protein or membrane protein complex is crosslinked using a crosslinking reagent that forms a linkage between a carbohydrate moiety and a thiol moiety, and in certain embodiments, the crosslinking reagent comprises: (i) N-beta-maleimidopropionic acid hydrazide (BMPH); or (ii) 3-(2-pyridyldithio)propionyl hydrazide (PDPH). In some embodiments, the membrane protein or membrane protein complex is capable of binding with a binding partner after crosslinking.

### EXAMPLES

The Examples that follow are illustrative of specific embodiments of the invention, and various uses thereof. They are set forth for explanatory purposes only, and are not to be taken as limiting the invention.

### Example 1

### Materials and Methods

Expression of hCXCR5:

Human CXCR5 receptor was expressed in Sf9 cells using the FastBac expression system (Invitrogen). Sf9 cells were grown in suspension in flasks with serum-free medium. Cells were infected with recombinant virus at a density of 6×10⁶ cells/ml; virus was added at the multiplicity of infection (MOI) of 10. An equal volume of fresh medium was added immediately afterward. Cells were harvested by centrifugation 72 h post-infection.

Purification of hCXCR5:

After expression Sf9 insect cell membranes were initially disrupted by homogenization in a hypotonic buffer containing 10 mM HEPES (pH 7.5), 20 mM KCl, and 10 mM MgCl2. Extensive washing of the isolated raw membranes was performed by repeated centrifugation (typically six to nine times) in a high-osmotic buffer containing 1.0 M NaCl, 10 mM HEPES (pH 7.5), 10 mM MgCl2, 20 mM KCl, and protease inhibitor cocktail (Roche), followed by Dounce homogenization to resuspend the membranes in fresh wash buffer thereby separating soluble and membrane-associated proteins from integral transmembrane proteins. Highly purified membranes were solubilized by incubation in the presence of 0.5% (w/v) n-dodecyl-D-maltopyranoside (DDM) (Sigma) and 0.01% (w/v) cholesteryl hemisuccinate (CHS) (Sigma) for two to three hours at 4° C. After solubilization, the unsolubilized material was removed by centrifugation at 150,000×g for 45 minutes. The supernatant was separated, supplemented with 25 mM buffered imidazole and incubated with Ni-NTA resin (Qiagen) overnight at 4° C.; typically, 1.5 ml of resin per one liter of original culture volume was used. After binding the resin was washed with ten column volumes of 25 mM HEPES (pH 7.5), 800 mM NaCl, 10% (v/v) glycerol, 55 mM imidazole, 0.05% (w/v) DDM and 0.001% (w/v) CHS. The receptor was eluted with 25 mM HEPES (pH 7.5), 800 mM NaCl, 10% (v/v) glycerol, 0.05% (w/v) DDM, 0.001% (w/v) CHS, and 200 mM imidazole. The eluted receptor was desalted with P-10 column (GE) to 25 mM HEPES (pH 7.5), 800 mM NaCl, 10% (v/v) glycerol, 0.05% (w/v) DDM, 0.001% (w/v) CHS and stored at −80° C.

Capturing hCXCR5 on Biacore Sensor Chip Surface:

For direct NTA chip capture, the purified hCXCR5 receptor was diluted 10 to 20-fold in HBS—N running buffer and injected with a flow rate of 5 μl/min to achieve capture level between 300-1000 resonance units (RU), depending on experiment purpose. For indirect CM5 chip capture, anti-6×His mAb (Qiagen) was amine-coupled to the free carboxyl groups on the chip surface using standard NHS/EDC coupling, yielding surface mAbs at about 15K RU. hCXCR5 receptor was then injected at 5 μl/min to achieve a similar capture level as for the NTA chip (i.e. between 300-1000 RU, depending on experiment purpose).

Stabilization of hCXCR5:

Direct- or indirect-captured hCXCR5 receptor protein was stabilized with crosslinking by treatment of mixtures of 0-200 μM EDC and 0-50 μM NHS for 7 min. Subsequently, the surface was deactivated with 1 M ethanolamine for 10 min. In separate experiments, five other crosslinkers, BS(PEG)₅ (bis(succinimidyl)penta(ethylene glycol)); DSS (disuccinimidyl suberate); SMCC (succinimidyl 4-(N-maleimidomethyl)cyclohexane-1-carboxylate); DTME (dithiobismaleimidoethane) and BM(PEG)₂ (1,8-bismaleimido-diethyleneglycol), all from Pierce, were tested at 50 μM concentration for stabilizing the captured hCXCR5 receptor at flow rate of 5 μl/min for 7 min. Excess crosslinkers were quenched with 1M ethanolamine for 10 min for amine reactive crosslinkers BS(PEG)₅ and DSS, or 50 mM cysteine in 0.1M sodium acetate and 1 M sodium chloride pH 4.0 for 10 min for thiol-reactive crosslinkers BM(PEG)₂ and DTME, or 1M ethanol amine for 10 min flowed with 50 mM cysteine in 0.1M sodium acetate and 1 M sodium chloride pH 4.0 for 10 min for heterobifunctional crosslinker SMCC. The surface stabilized receptor chip was primed with running buffer before subsequent assay steps.

Regeneration of Captured-Stabilized hCXCR5 Surfaces:

At the end of each binding cycle, the GPCR surfaces were regenerated with injection of 50 mM HCl for 1 min.

Kinetic Characterization of hCXCR5 Ligand and Antibodies:

CXCL13 and 3 anti-hCXCR5 mAbs were each tested in triplicate in 2-fold dilution series for binding to hCXCR5 in HBS-EP⁺ running buffer. All samples were injected at a flow rate of 50 μl/min.

Data Processing and Analysis:

All biosensor data processing and analysis was performed using Biacore T100 Evaluation Software (GE). For kinetic analyses, data were locally fit to a 1:1 interaction model. CV (coefficient of variation) was calculated as the standard deviation (STDEV) divided by the mean.

### Example 2

### Conventional Peptide-Based Biacore Assay

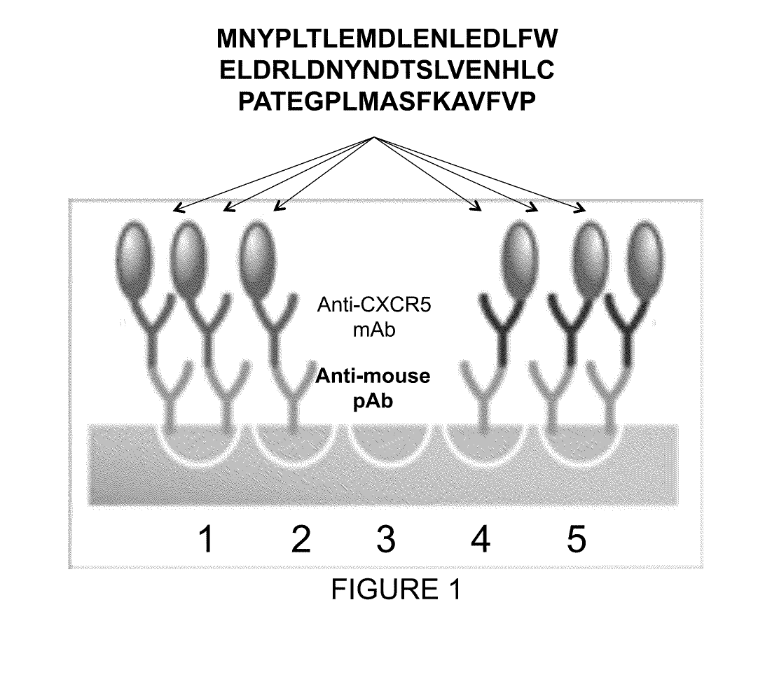

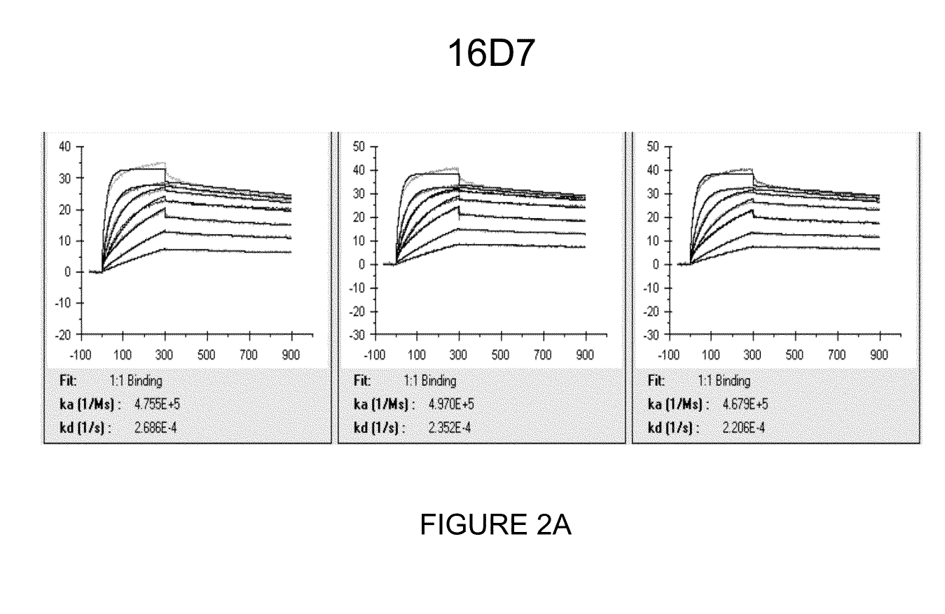

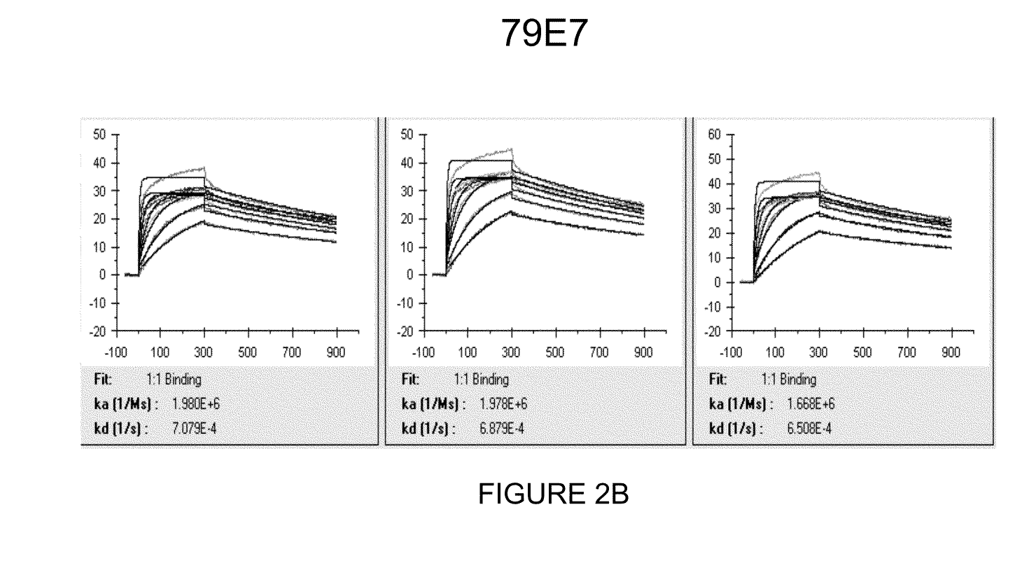

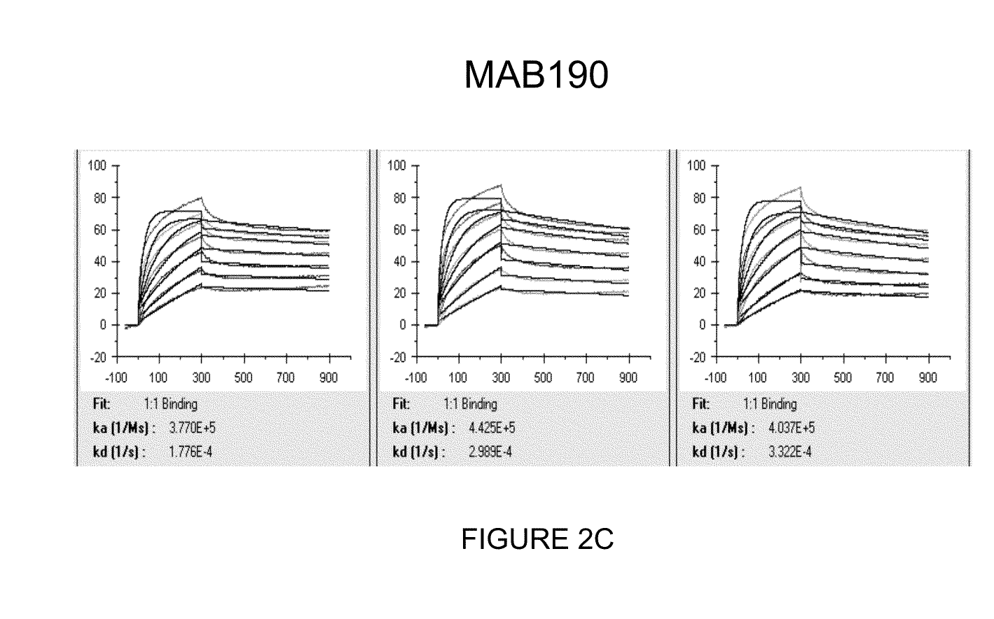

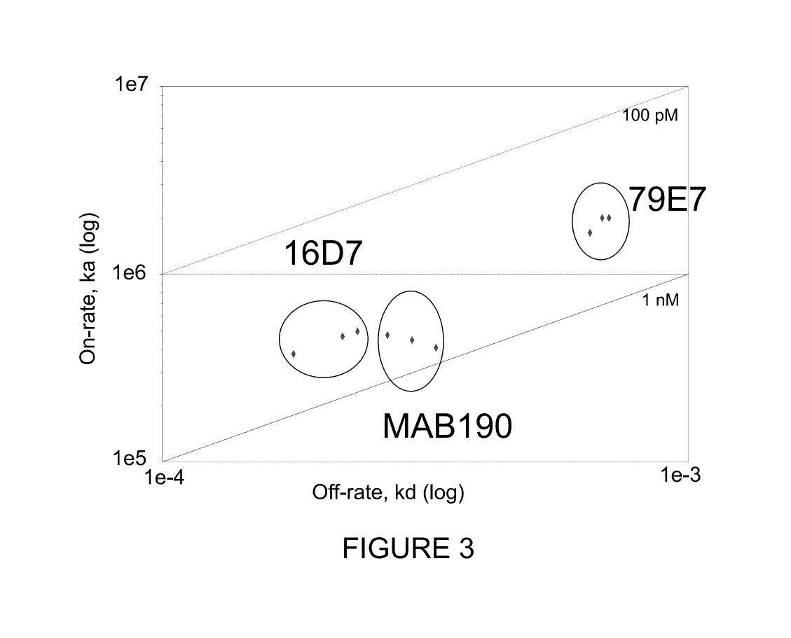

A mouse antibody capturing kit from GE was used for Biacore CM5 chip surface preparation. Following standard protocols provided by the manufacturer, as shown in FIG. 1, the anti-mouse polyclonal antibody was first immobilized on the chip surface via amine coupling to the free carboxyl groups on the CM5 chip surface using standard NHS/EDC procedures. Mouse antibodies against hCXCR5 were then captured by the anti-mouse antibody. Next, the hCXCR5 N-terminal peptide (MNYPLTLEMD LENLEDLFWE LDRLDNYNDT SLVENHLCPA TEGPLMASFK AVFVP) (SEQ ID NO: 1) was injected over the captured anti-hCXCR5 antibodies at various concentrations to generate a kinetics sensorgram as shown in FIG. 2. This assay format had good reproducibility as seen in Table 1. However, it did not provide sufficient resolution and sensitivity to differentiate the 3 mAb clones which is reflected in the Kₒₙ/K_{off} rate map shown in FIG. 3.

TABLE 1
Biacore kinetics of 3 mAb clones against hCXCR5
using the conventional peptide based method.

| Sample | | ka (1/Ms) | kd (1/s) | KD (M) |
|---|---|---|---|---|
| 16D7 | Mean | 4.80E+05 | 2.41E−04 | 5.03E−10 |
| | CV | 3.14E−02 | 1.02E−01 | 1.06E−01 |
| 79E7 | Mean | 1.88E+06 | 6.82E−04 | 3.65E−10 |
| | CV | 9.58E−02 | 4.25E−02 | 6.09E−02 |
| MAB190 | Mean | 4.08E+05 | 2.70E−04 | 6.56E−10 |
| | CV | 8.08E−02 | 3.02E−01 | 2.69E−01 |

### Example 3

### Reversed Peptide-Based Biacore Assay

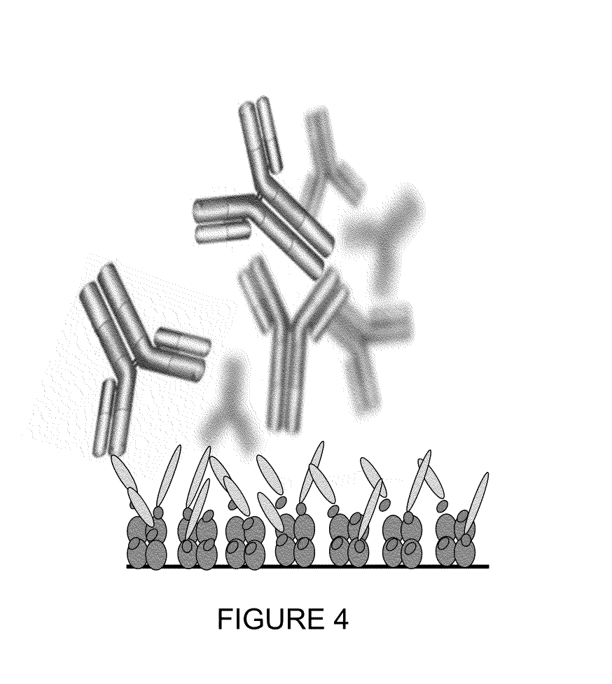

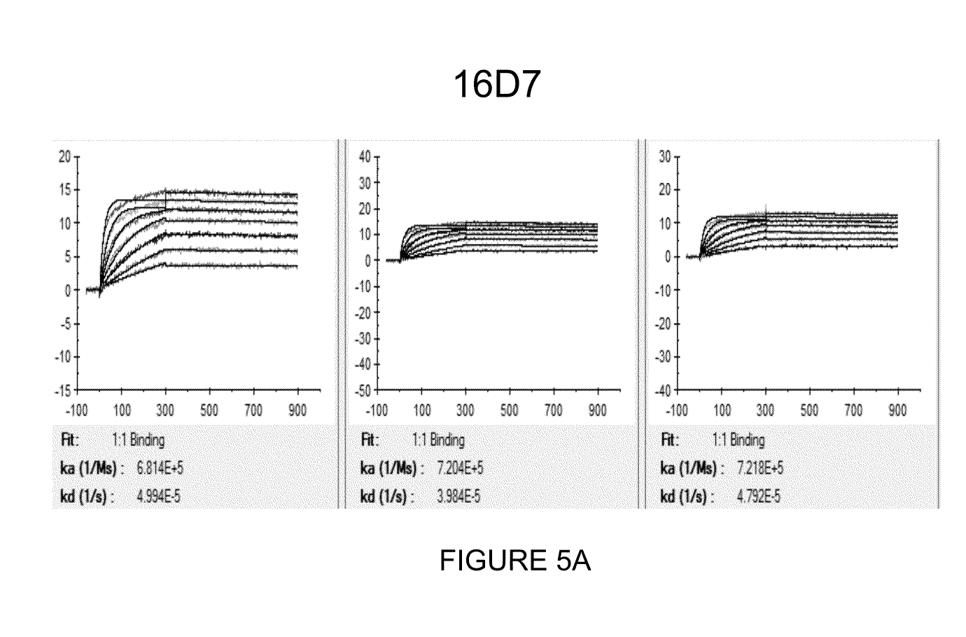

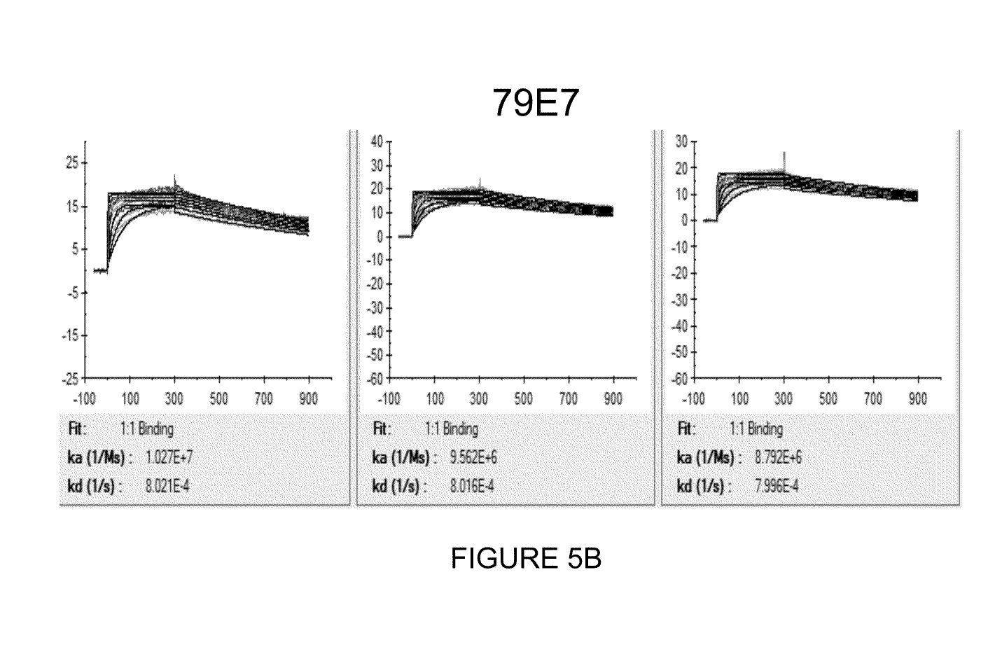

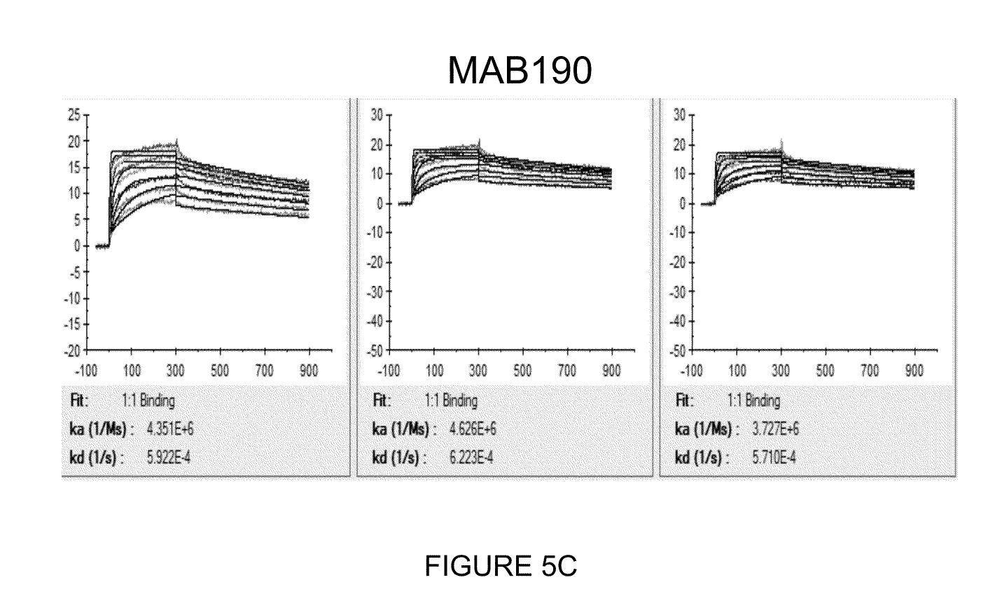

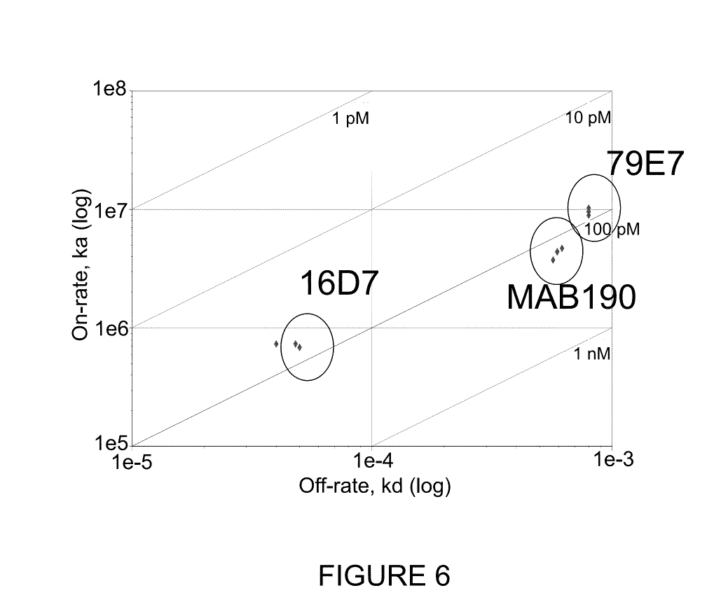

This assay format took advantage of the availability of biotinylated N-terminal hCXCR5 peptide and a streptavidin (SA) Biacore chip. As shown in FIG. 4, biotinylated peptide was captured by streptavidin at low density. Unused biotin-binding sites were blocked by free biotin. Anti-hCXCR5 monoclonal antibodies generated against the N-terminal peptide as an immunogen were injected at various concentrations to generate Biacore kinetics sensorgrams as shown in FIG. 5. In this assay format, clone 16D7 showed more then 10-fold slower off-rate (Table 2), which was not detectable by the conventional assay shown in Table 1 and FIG. 3. The Kₒₙ/K_{off} rate map (FIG. 6) also indicated that this reversed peptide-based assay format displayed good reproducibility.

TABLE 2
Biacore kinetics of 3 mAb clones against hCXCR5
using the reversed format of peptide based assay.

| Sample | | ka (1/Ms) | kd (1/s) | KD (M) |
|---|---|---|---|---|
| 16D7 | Mean | 7.08E+05 | 4.59E−05 | 6.50E−11 |
| | CV | 3.24E−02 | 1.16E−01 | 1.40E−01 |
| 79E7 | Mean | 9.54E+06 | 8.01E−04 | 8.43E−11 |
| | CV | 7.74E−02 | 1.63E−03 | 7.63E−02 |
| MAB190 | Mean | 4.23E+06 | 5.95E−04 | 1.41E−10 |
| | CV | 1.09E−01 | 4.33E−02 | 7.33E−02 |

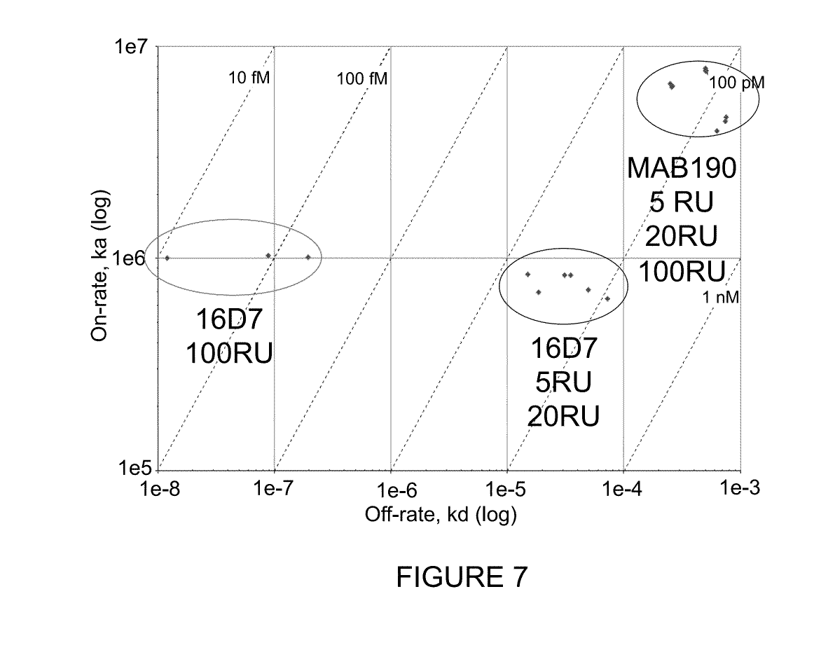

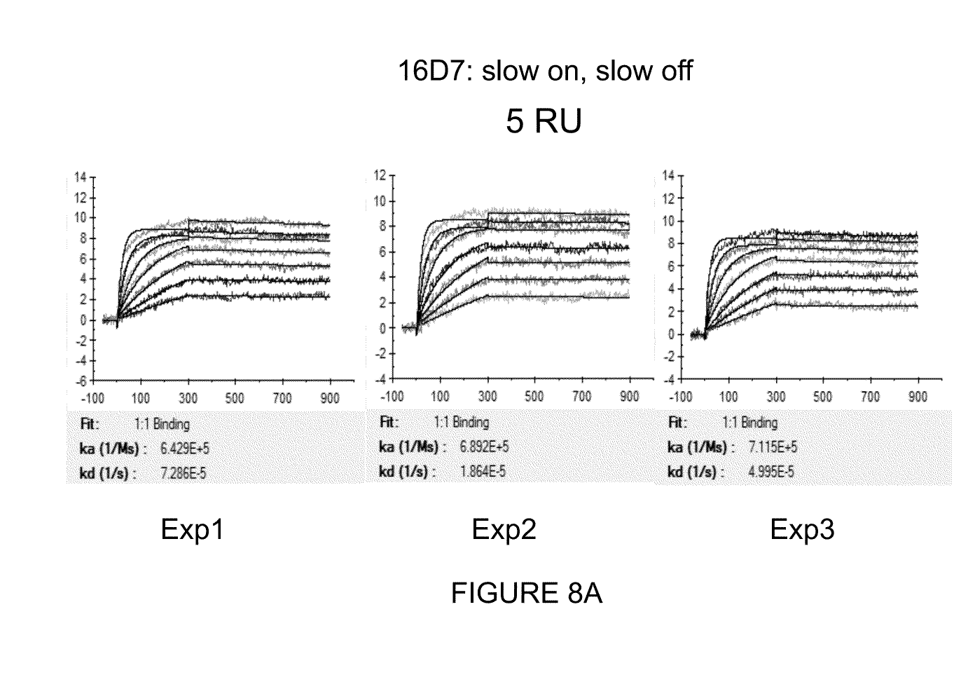

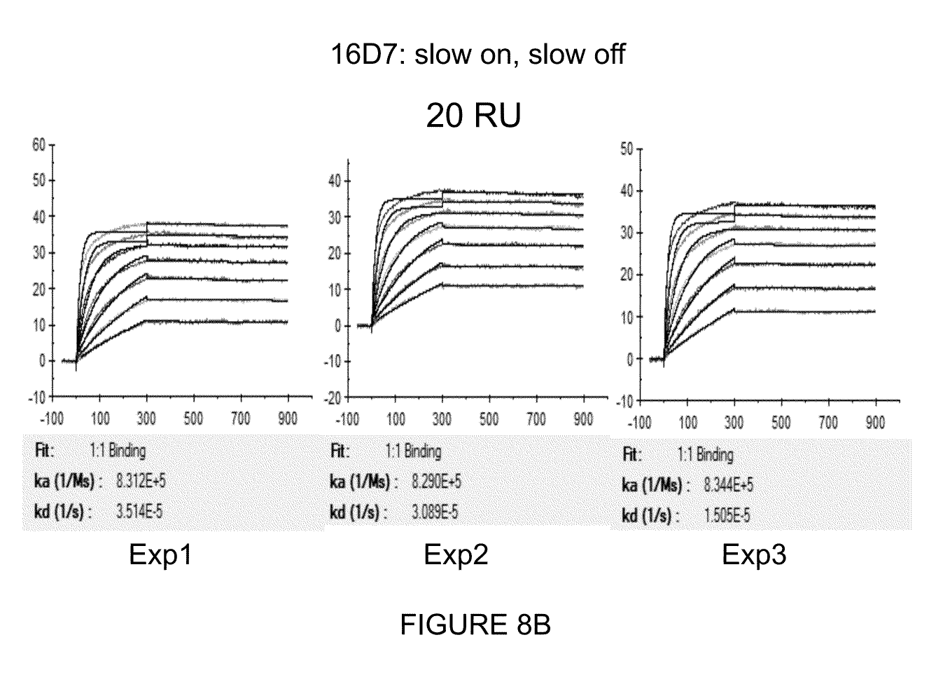

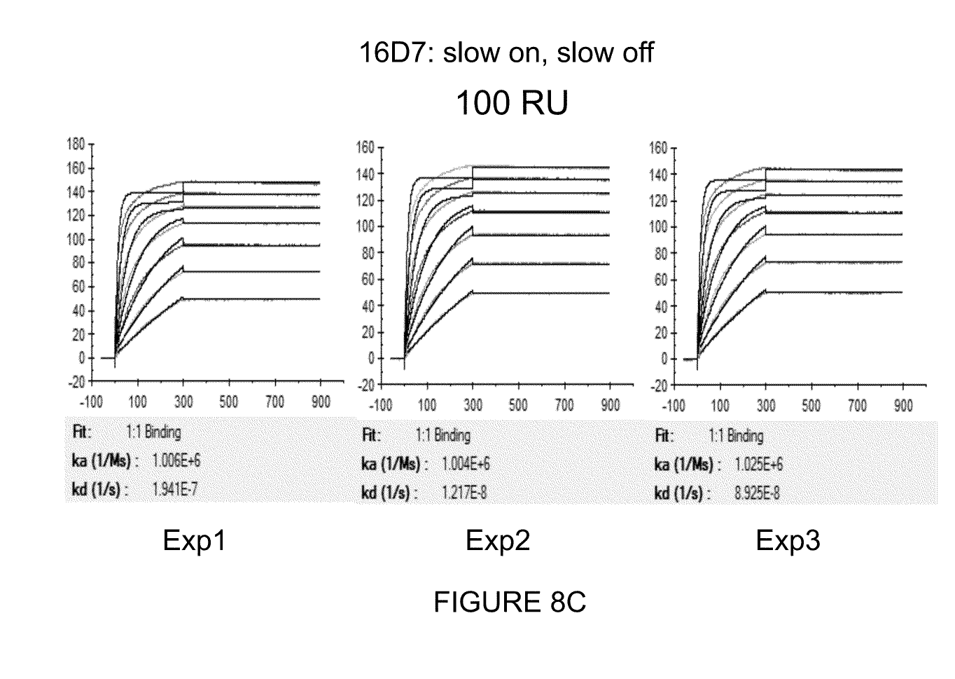

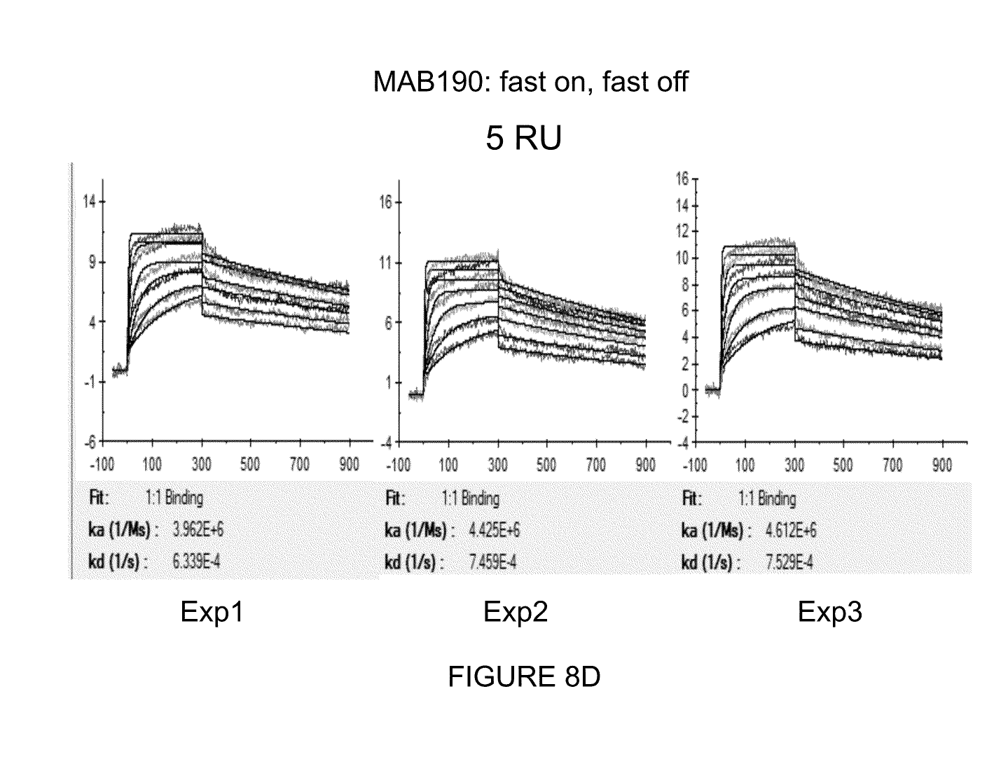

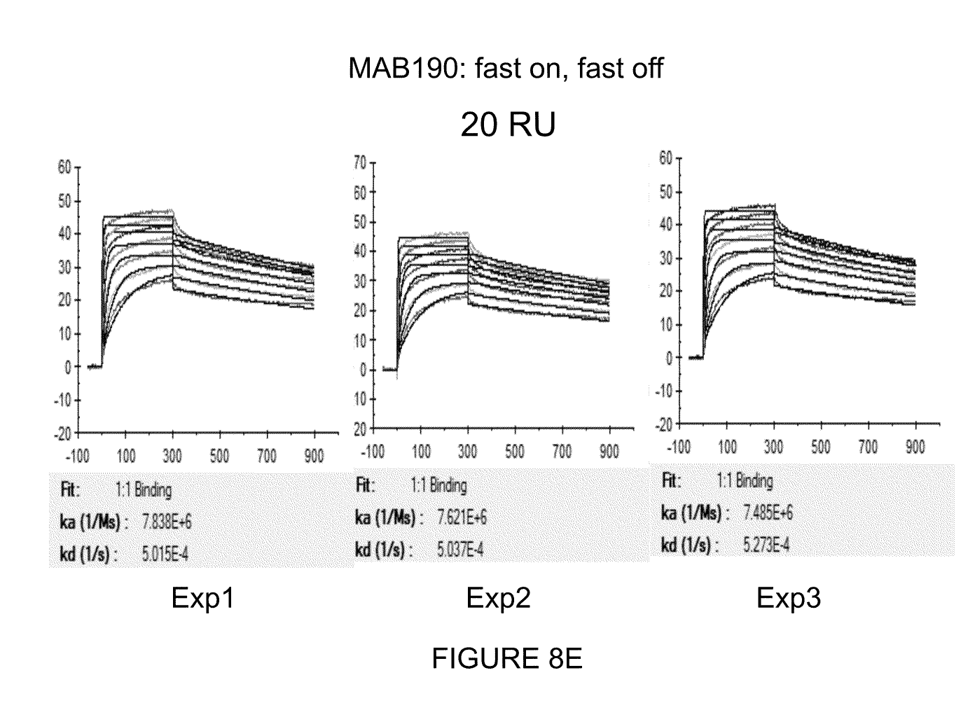

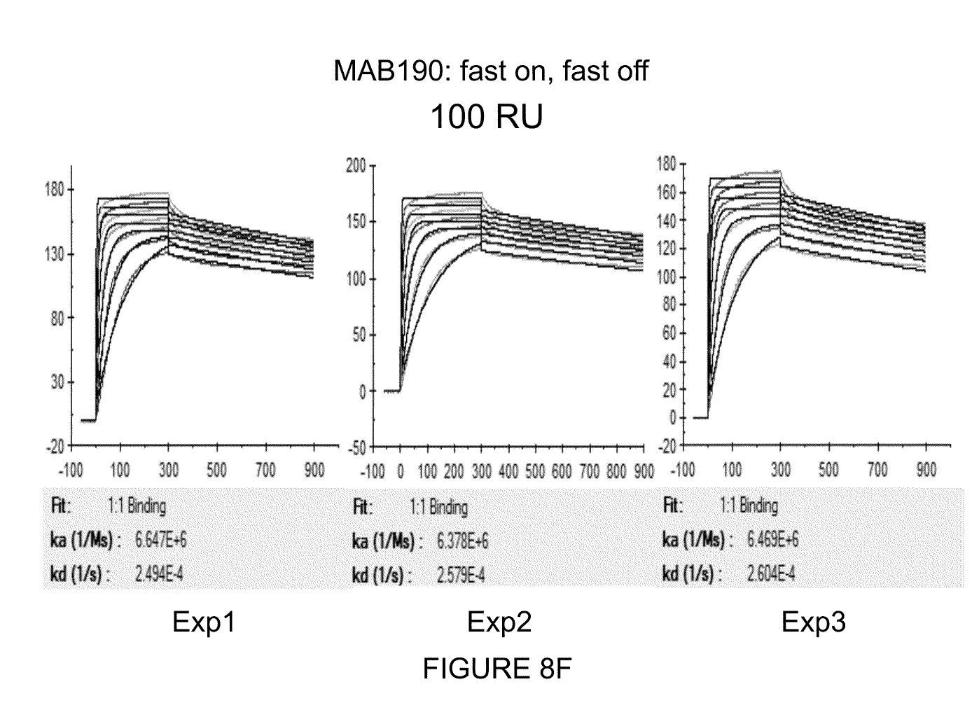

However, since antibodies are dimeric molecules, it is possible that one antibody molecule could bind to two peptides on the chip surface thereby resulting in artificially high affinity through the avidity effect. In fact, affinity (KD) data generated by this assay format indeed was higher than the data generated by the conventional assay format. Therefore, the higher affinity data for the same antibody at least in part is attributed to the avidity effect. In order to evaluate the avidity effect, different levels (5, 20, and 100 RU) of biotinylated peptides were captured on a SA chip surface and kinetics assays were performed using the same procedures as above. As shown in FIG. 7, although the overall antibody binding RUs correlated to the peptide level captured, the shape of the sensorgrams for each antibody at different peptide concentrations were similar. As shown in FIG. 8, at three different peptide levels, MAB190 showed similar affinity results whereas clone 16D7 was dramatically different. The high level of peptide (100RU) did indeed alter the overall affinity (KD), mainly due to artificial K_{off} (9.85×10⁻⁸, Table 3), which was outside of the Biacore accuracy limit.

TABLE 3
Comparison of peptide levels used in the
reversed format of peptide based assay.

| mAb | Peptide Density | | ka (1/Ms) | kd (1/s) | KD (M) |
|---|---|---|---|---|---|
| 16D7 | 5RU | Mean | 6.81E+05 | 4.71E−05 | 7.02E−11 |
| | | CV | 5.14E−02 | 5.77E−01 | 6.15E−01 |
| | 20RU | Mean | 8.32E+05 | 2.70E−05 | 3.25E−11 |
| | | CV | 3.26E−03 | 3.92E−01 | 3.93E−01 |
| | 100RU | Mean | 1.01E+06 | 9.85E−08 | 9.74E−14 |
| | | CV | 1.13E−02 | 9.27E−01 | 9.33E−01 |
| MAB190 | 5RU | Mean | 4.33E+06 | 7.11E−04 | 1.64E−10 |
| | | CV | 7.72E−02 | 9.39E−02 | 2.64E−02 |
| | 20RU | Mean | 7.65E+06 | 5.11E−04 | 6.68E−11 |
| | | CV | 2.33E−02 | 2.80E−02 | 4.93E−02 |
| | 100RU | Mean | 6.50E+06 | 2.56E−04 | 3.94E−11 |
| | | CV | 2.10E−02 | 2.27E−02 | 4.15E−02 |

### Example 4

### hCXCR5 Whole-Receptor Biacore Assay

Peptide-based assays for anti-GPCR antibodies are convenient; however, they can only be used for analyzing antibodies recognizing linear epitopes. The conventional assay format (Example 2) lacks resolution (it can't distinguish three mAb clones) whereas the reversed assay format (Example 3) lacks accuracy due to the avidity effect. A whole-receptor assay addresses these issues.

The whole-receptor assay method requires mutagenesis to obtain a stable GPCR molecule and it relies on utilizing a low-affinity small molecule compound for regeneration. Therefore, it is not applicable to antibody analysis. Nevertheless, a simple yet reliable method for analyzing antibodies against GPCRs was developed.

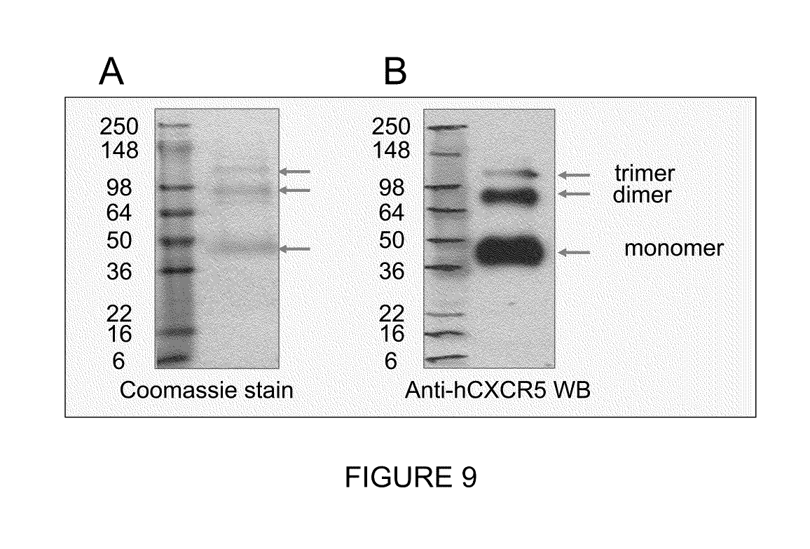

As described in Example 1, C-terminal 6×His-tagged hCXCR5 whole receptor protein was expressed in insect cells and purified via IMAC affinity purification as shown in FIG. 9. The purified protein was composed of monomers as well as dimers and trimers as observed by SDS-PAGE and Western blot analysis. See FIG. 9. The final purified receptor protein was desalted to a membrane protein storage buffer with the composition of 25 mM HEPES (pH 7.5), 800 mM NaCl, 10% (v/v) glycerol, 0.05% (w/v) DDM, 0.001% (w/v) cholesteryl hemisuccinate.

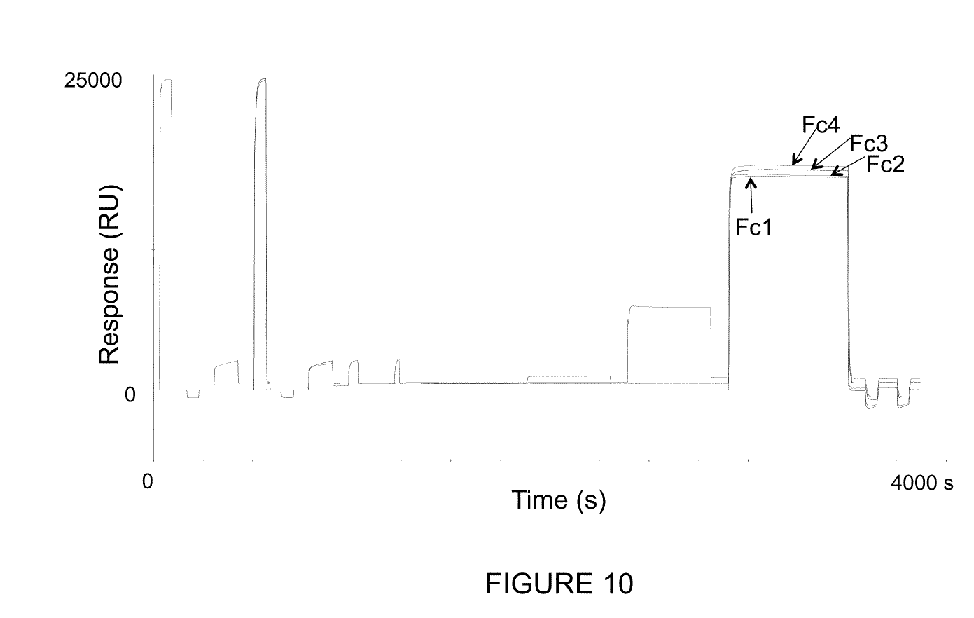

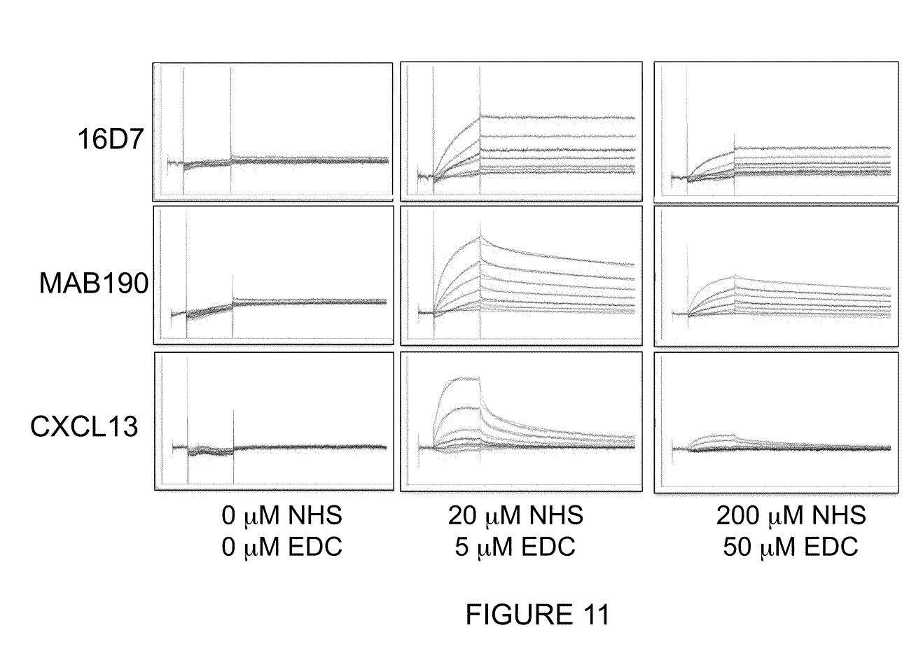

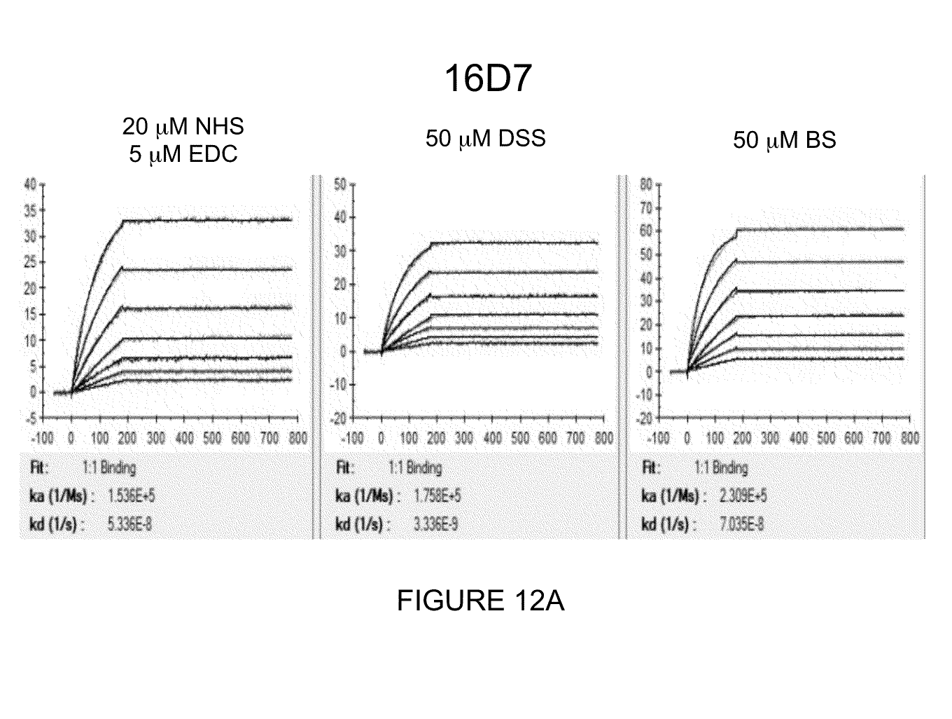

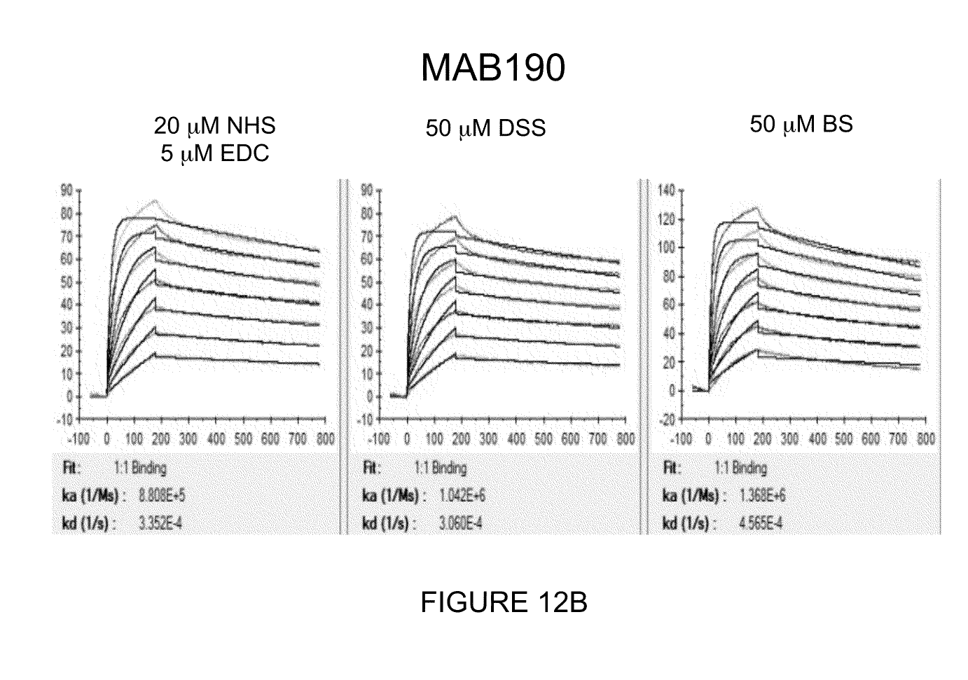

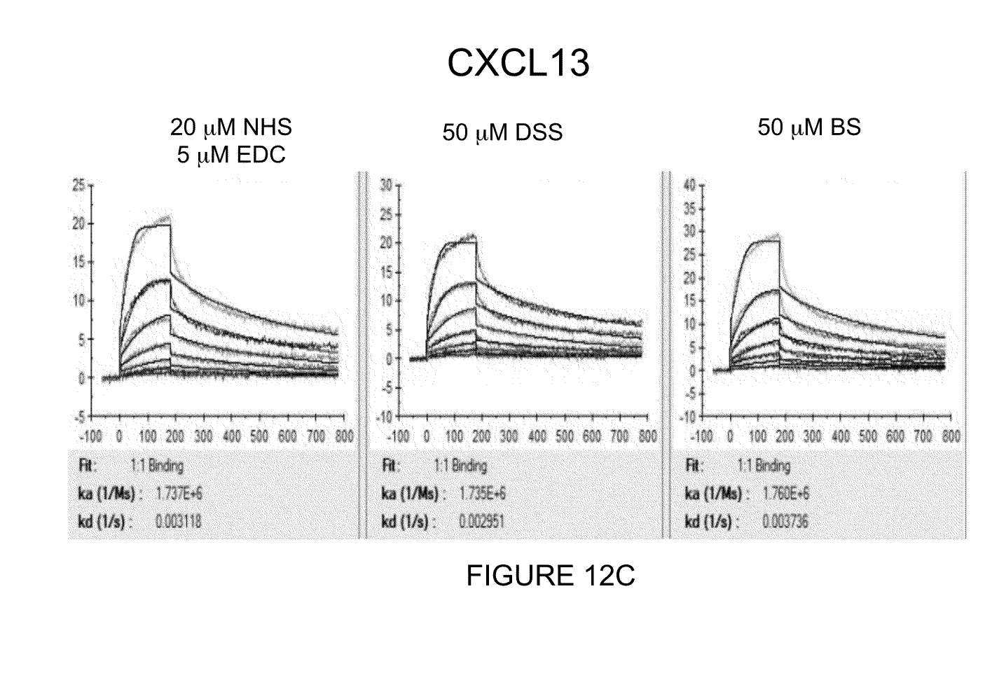

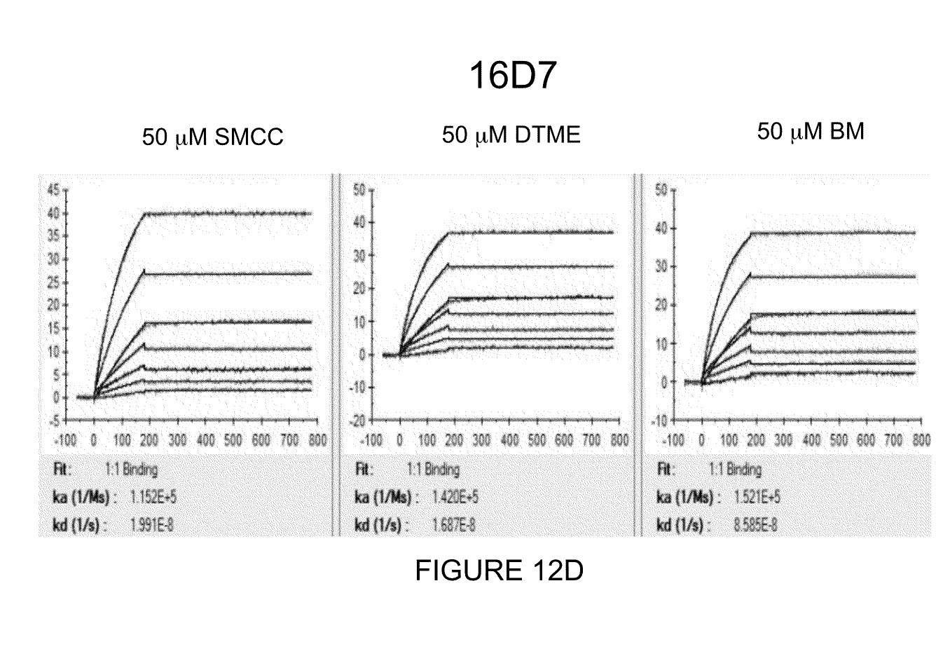

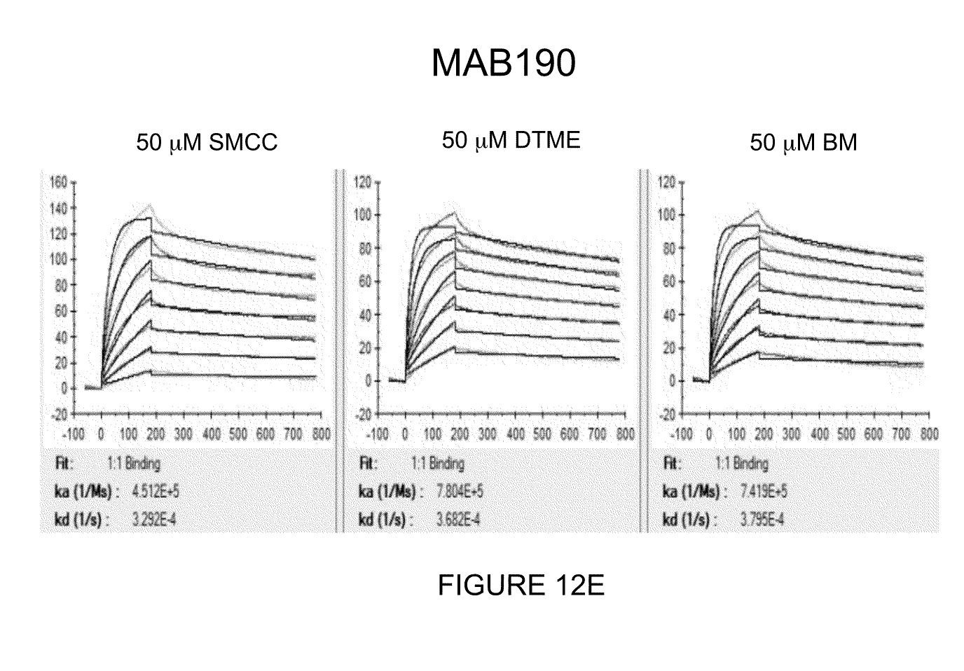

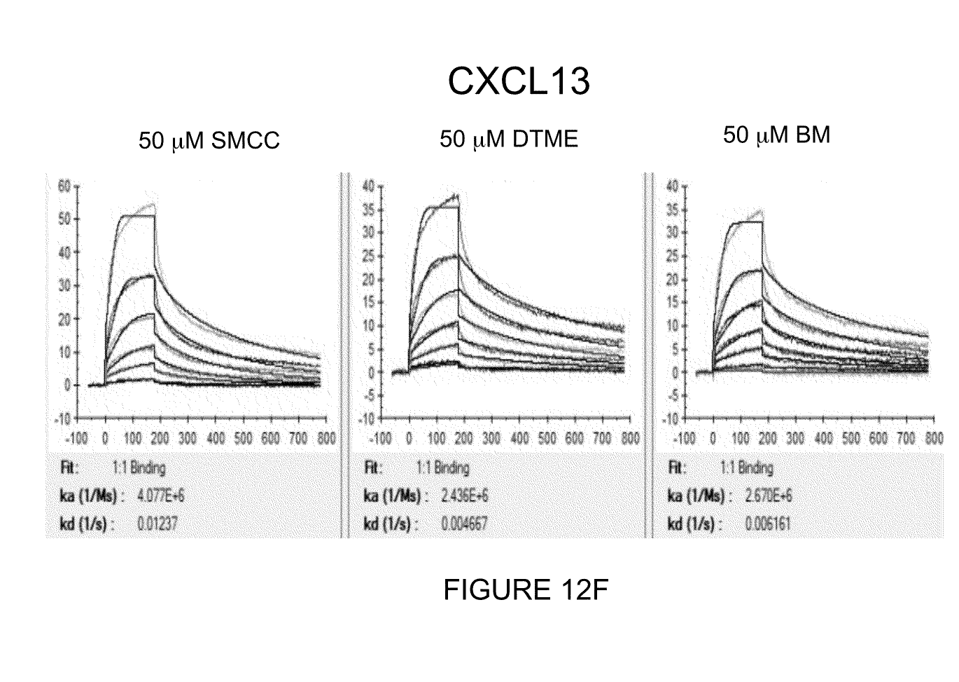

For direct capturing by an NTA SPR sensor chip, the purified 6×His-hCXCR5 protein was diluted to 50 ug/ml with HBS—N (from GE) buffer. An NTA reagent kit (GE) was used for chip surface preparation. The flow rate was set to 5 ul/ml and HBS—N was used as the running buffer. As shown in FIG. 10, designated flow cells were first pretreated with 350 mM EDTA for 1 min, then activated with 0.5 mM NiCl₂ for 1 min. the diluted receptor protein was then injected to reach the required RU (300-1000 RUs depending on the purposes). In order to stabilize native-like structure of the receptor captured on the chip surface, the commonly used amine coupling reagent NHS/EDC was tested at different concentrations as shown in FIGS. 11 and 12. Among three tested concentrations, 20 μM NHS and 5 μM EDC gave the optimal results. As expected, without stabilization, the Ni-NTA bound 6×His-hCXCR5 protein was not stable and as a result could not survive the regeneration step (50 mM HCl for 1 min). Higher concentrations of the crosslinking reagent (200 μM NHS/50 μM EDC) caused damage to the receptor binding sites for antibody binding.

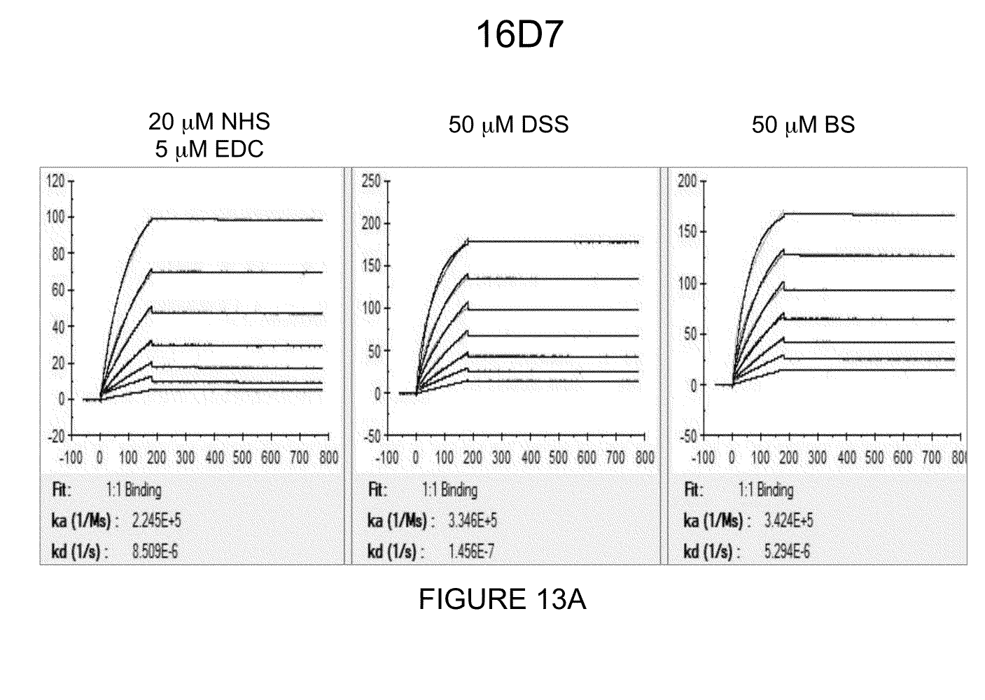

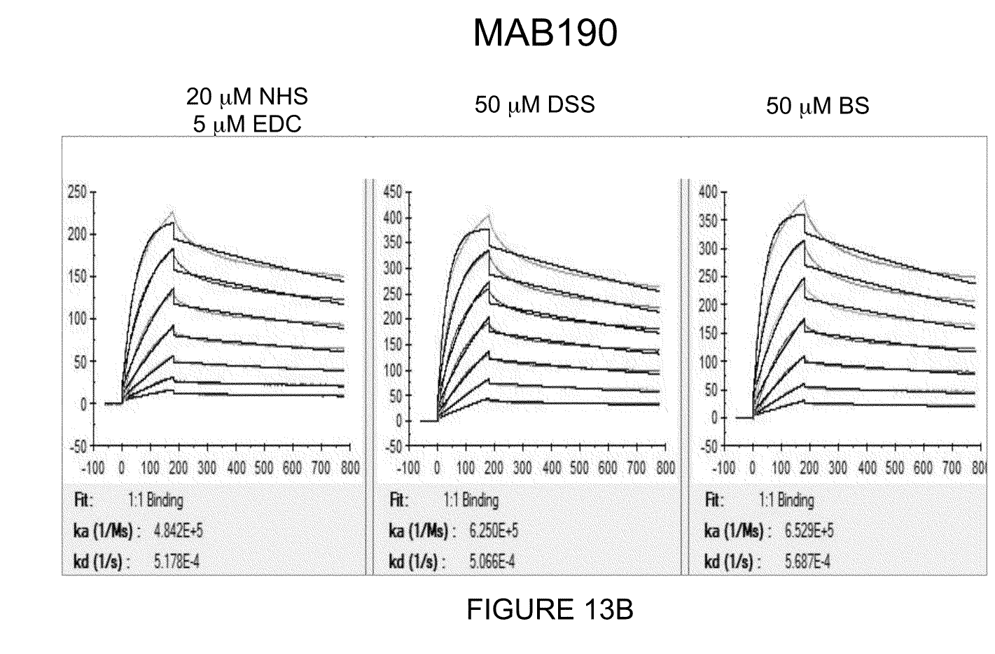

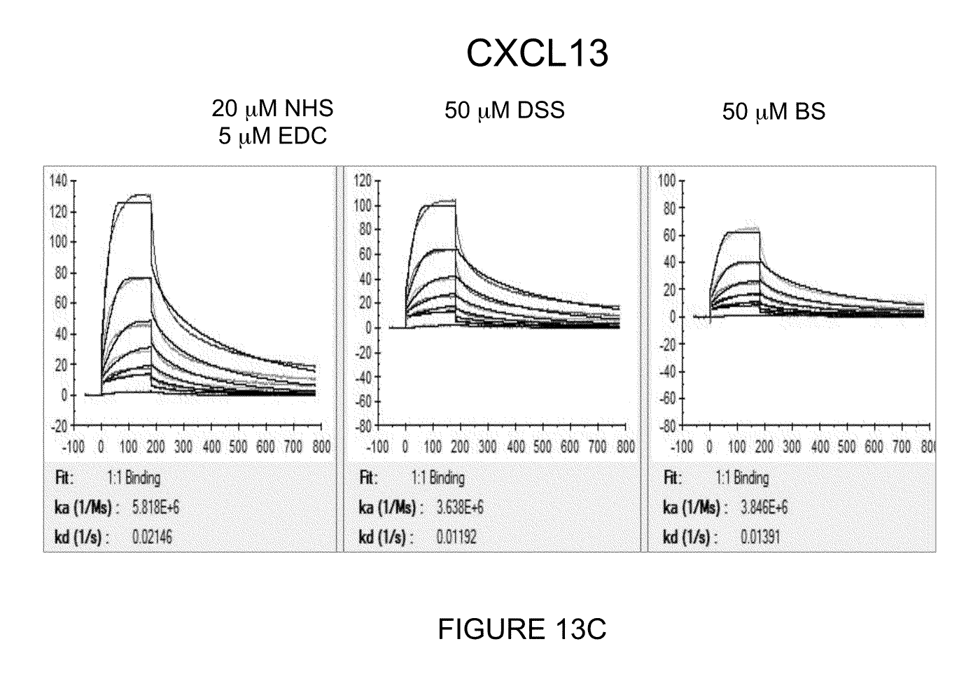

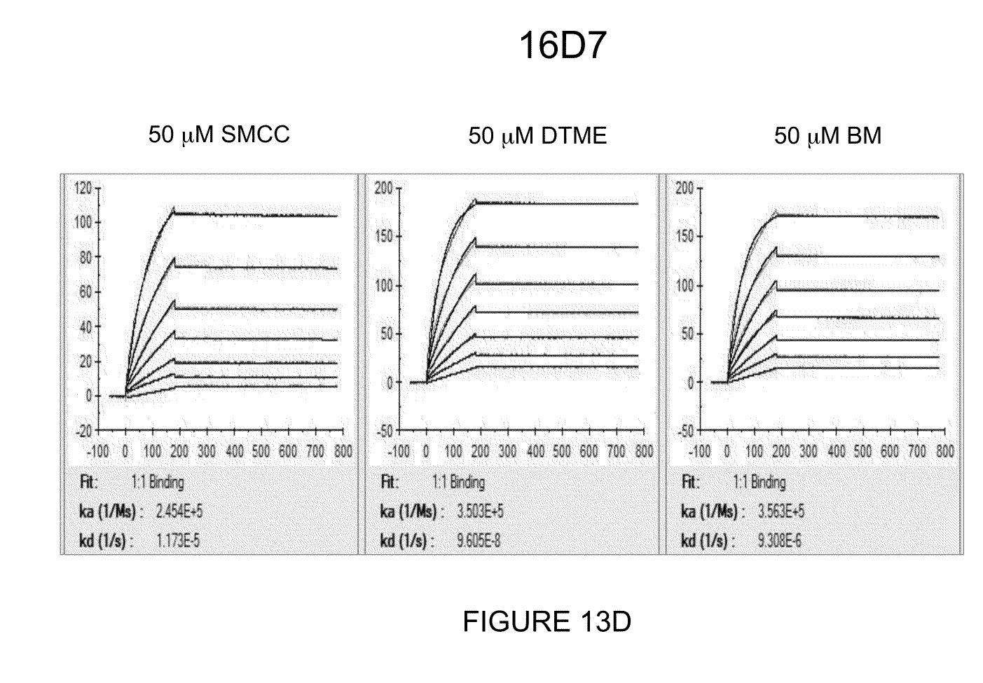

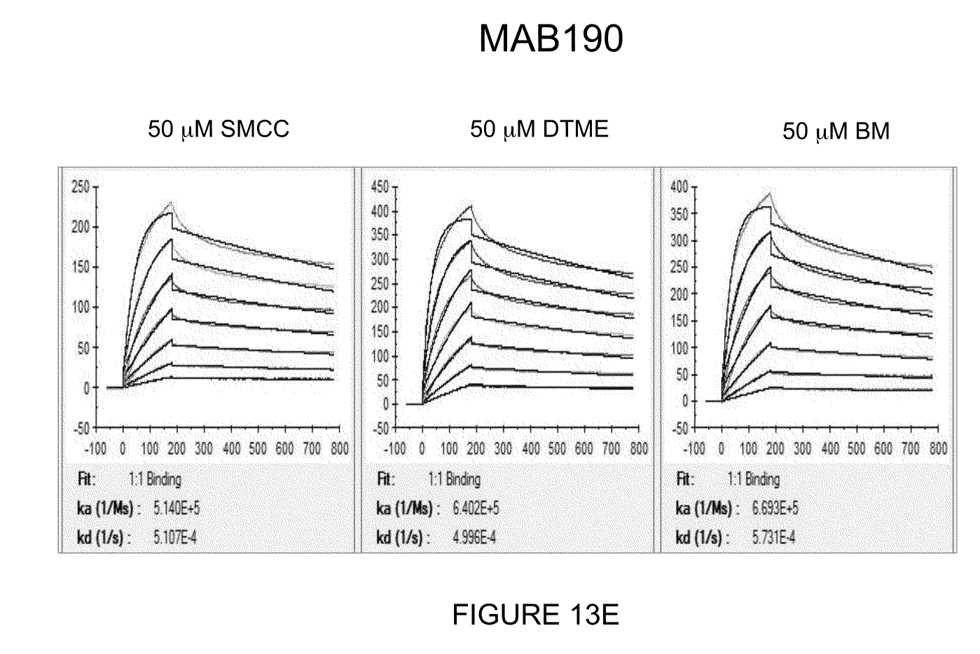

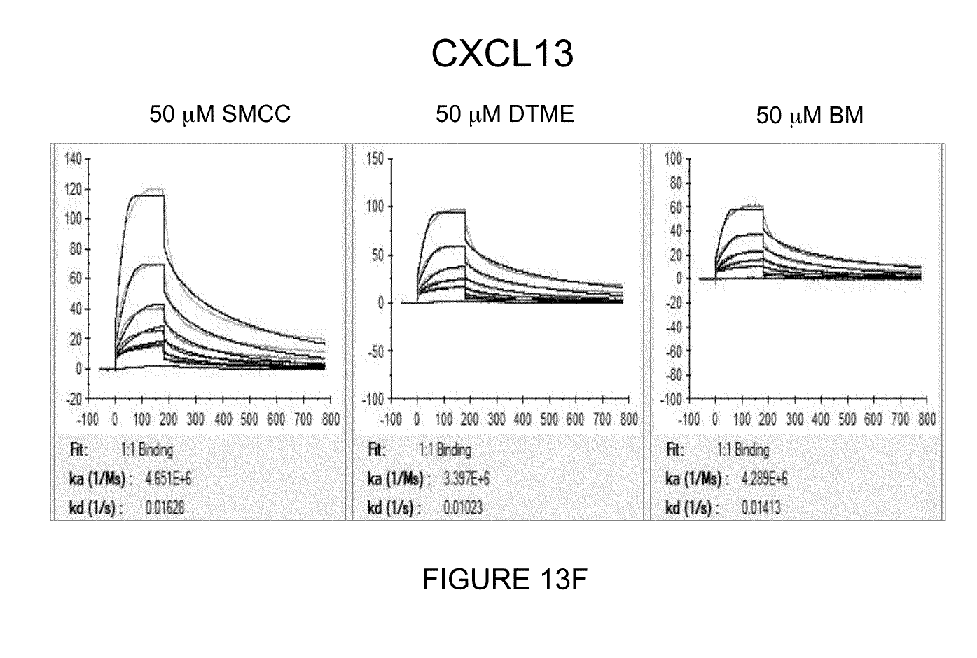

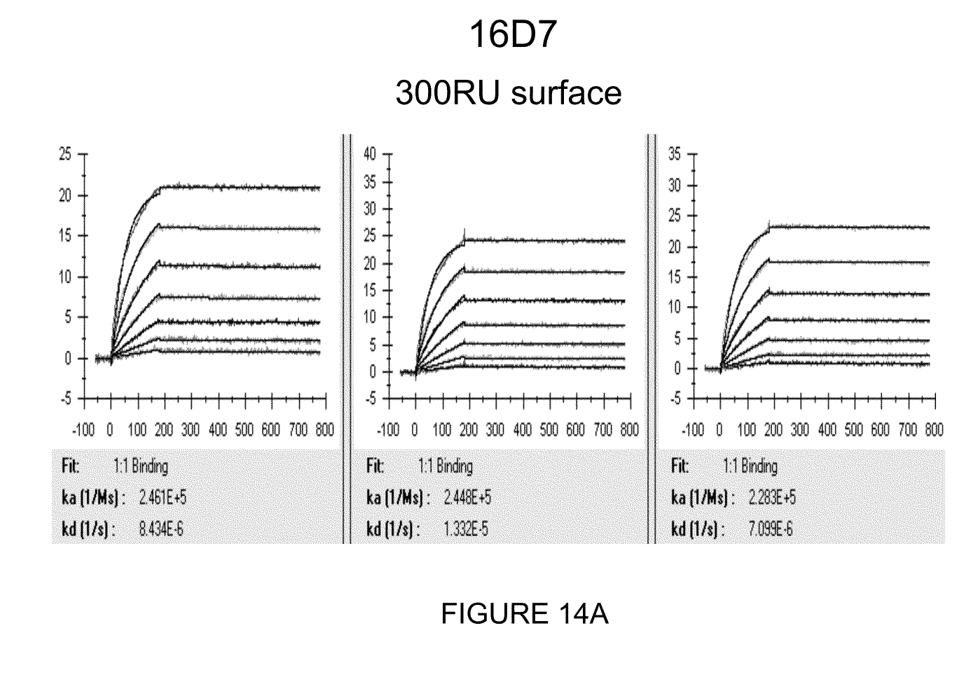

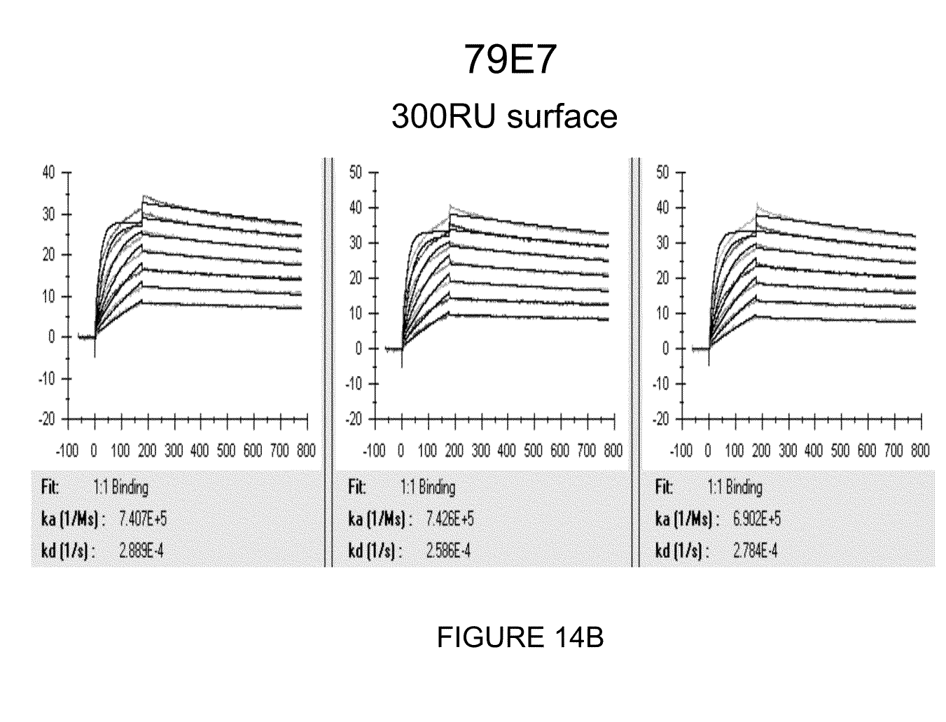

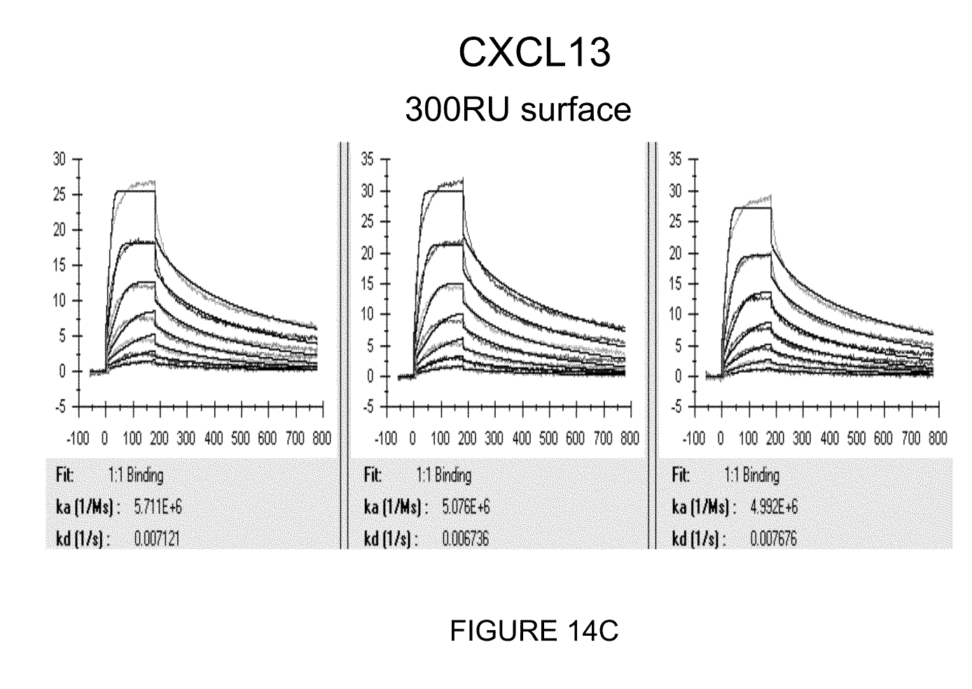

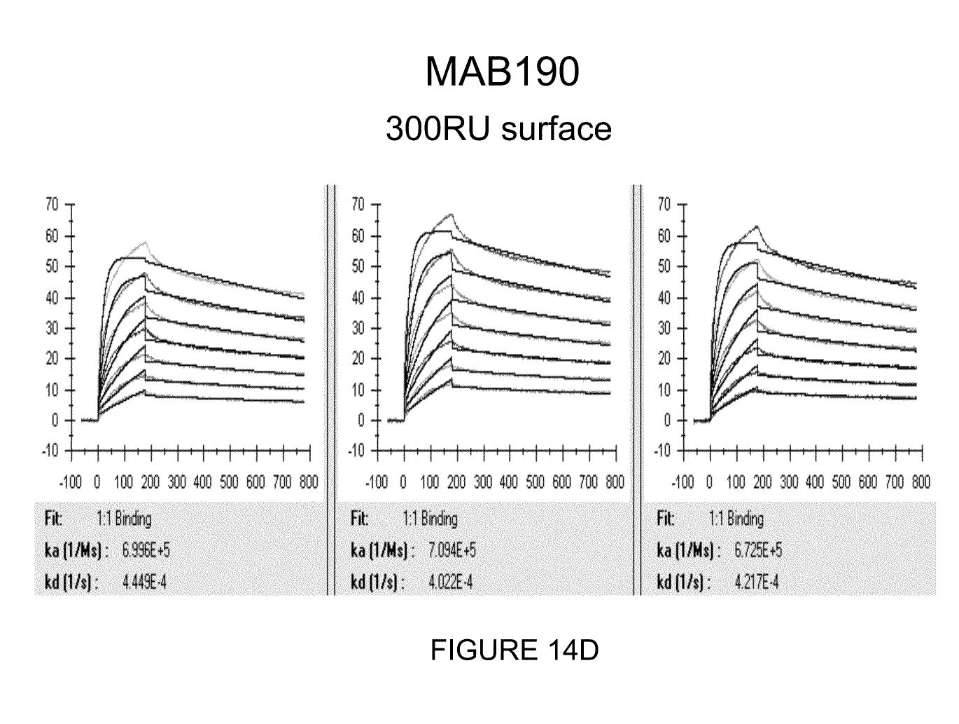

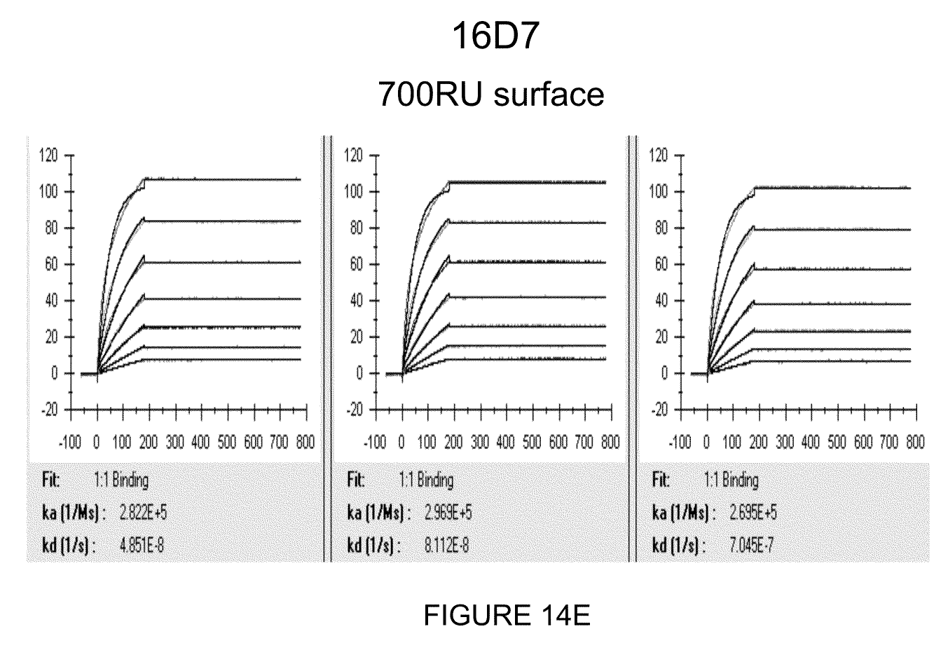

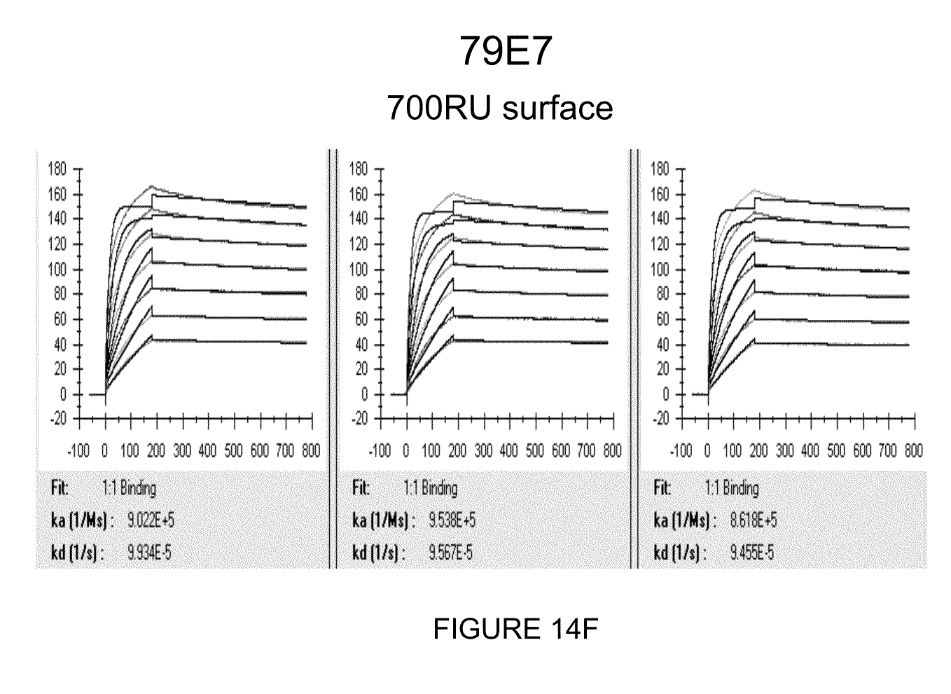

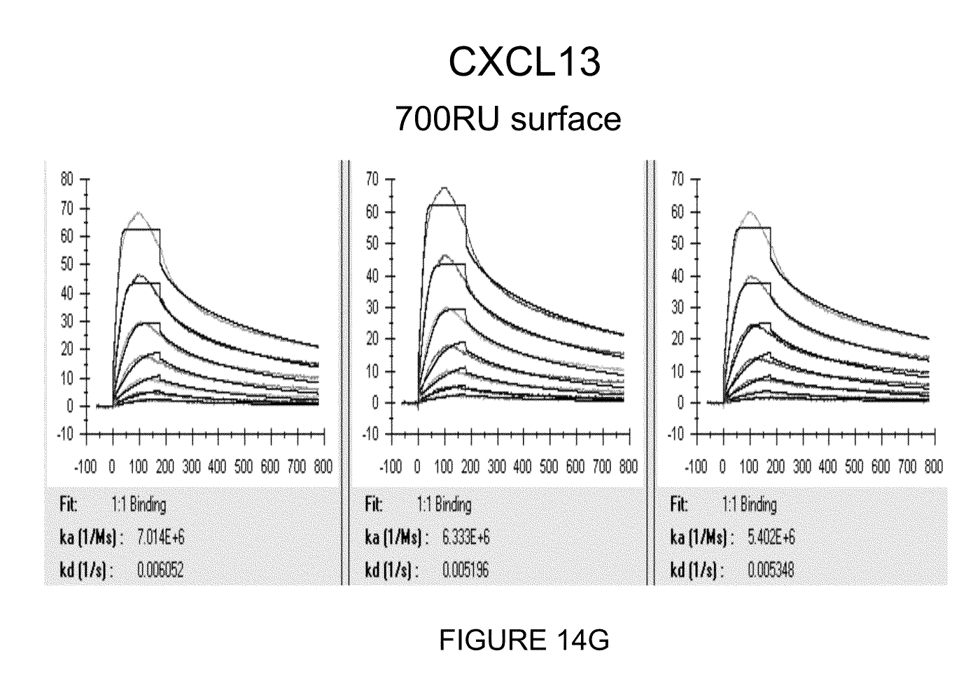

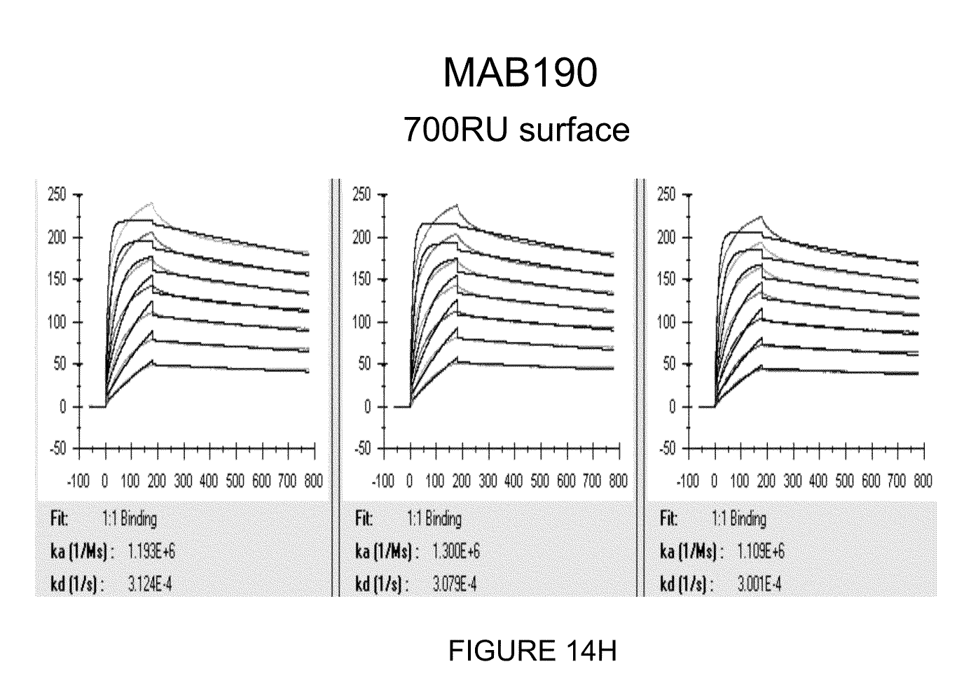

Similarly, the purified 6×His-hCXCR5 protein was captured via anti-6×His antibody which was immobilized on CM5 chip surface by a standard antibody coupling method. Five other crosslinkers were evaluated as shown in FIGS. 12 and 13 and as discussed in Example 1. The crosslinked membrane receptor proteins were also tested for the effect on structural stabilization post capturing on both NTA and CM5 sensor chips. All of the crosslinkers showed similar capability for receptor stabilization as compared to NHS/EDC on both chip surfaces.

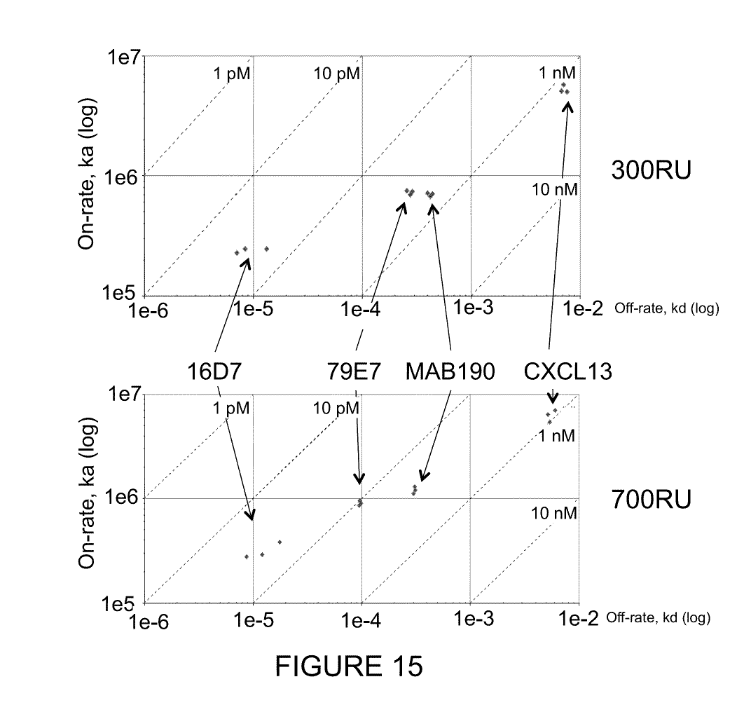

In order to assess the impact of the avidity effect on this capture and stabilization method, chip surfaces with different receptor densities were prepared. As shown in FIG. 15, high quality kinetics sensorgrams were generated with good reproducibility. The Kₒₙ/K_{off} rate map further highlighted the reproducibility of triplicate data points. Three antibody samples as well as the ligand were well-differentiated by this assay format, which was not possible using the two other peptide-based assay formats (discussed in Examples 2 and 3). The kinetics data shown in Table 4 further demonstrated the reliability of the assay regardless the difference in receptor density. Therefore, this limited chemical crosslinking step generated a stable sensor chip surface with full ligand binding activity. Using this method, the stabilized chip surface can be regenerated with 50 mM HCl for thousands of cycles without a noticeable or significant loss of binding activity.

TABLE 4
Comparison of hCXCR5 whole receptor levels
captured and stabilized on NTA chip surface.

| Receptor | | | | | |
|---|---|---|---|---|---|
| Density | Sample | | ka (1/Ms) | kd (1/s) | KD (M) |
| 300RU | 16D7 | Mean | 2.40E+05 | 9.62E−06 | 3.99E−11 |
| | | CV | 4.14E−02 | 3.40E−01 | 3.17E−01 |
| | 79E7 | Mean | 7.24E+05 | 2.75E−04 | 3.81E−10 |
| | | CV | 4.11E−02 | 5.60E−02 | 7.57E−02 |
| | CXCL13 | Mean | 5.26E+06 | 7.18E−03 | 1.37E−09 |
| | | CV | 7.47E−02 | 6.59E−02 | 1.10E−01 |
| | MAB190 | Mean | 6.94E+05 | 4.23E−04 | 6.10E−10 |
| | | CV | 2.75E−02 | 5.05E−02 | 6.15E−02 |
| 700RU | 16D7 | Mean | 3.18E+05 | 1.28E−05 | 3.96E−11 |
| | | CV | 1.83E−01 | 3.53E−01 | 1.91E−01 |
| | 79E7 | Mean | 9.06E+05 | 9.65E−05 | 1.07E−10 |
| | | CV | 5.09E−02 | 2.59E−02 | 5.20E−02 |
| | CXCL13 | Mean | 6.25E+06 | 5.53E−03 | 8.91E−10 |
| | | CV | 1.29E−01 | 8.25E−02 | 9.90E−02 |
| | MAB190 | Mean | 1.20E+06 | 3.07E−04 | 2.56E−10 |
| | | CV | 7.99E−02 | 2.03E−02 | 6.85E−02 |

### Example 5

### Anti-CD52 Antibody Analysis by VLP Captured on Biacore Sensor Chip and Stabilized with Limited Chemical Crosslinking

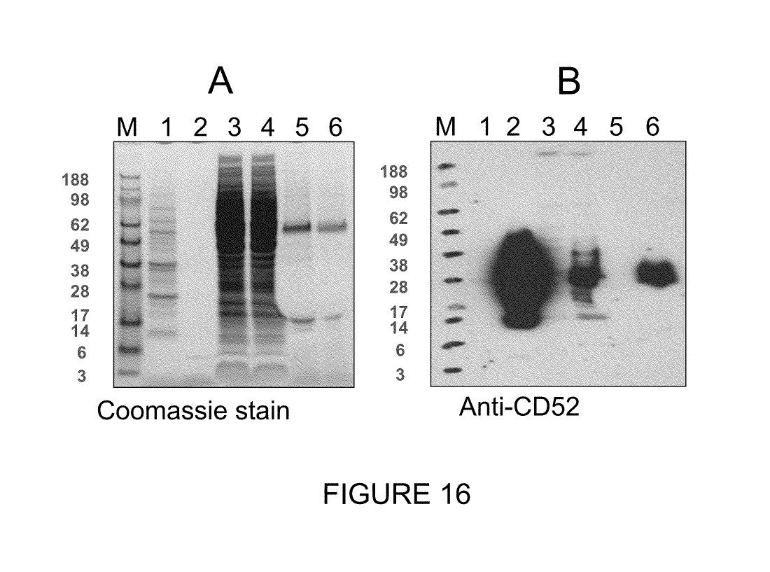

To further expand the applicability of limited chemical crosslinking for non-purified protein samples, a virus-like particle (VLP) expression system was developed. Full-length CD52 coding sequence (ATGAAGCGCTTCCTCTTCCTCCTACTCACCA TCAGCCTCCTGGTTATGGTACAGATACAAACTGGACTCTCAGGACAAAACGACACC AGCCAAACCAGCAGCCCCTCAGCATCCAGCAGCATGAGCGGAGGCATTTTCCTTTT CTTCGTGGCCAATGCCATAATCCACCTCTTCTGCTTCAGTTGA) (SEQ ID NO: 2) was cloned into pEF_DEST51 vector (Invitrogen). Following the user guide for MembranePro™ Functional Protein Expression System from Life Technologies, CD52 VLP prep was obtained. As shown in FIG. 16, SDS-PAGE and Western blot analysis showed the presence of CD52 in both the total cell lysates and in the VLP prep.

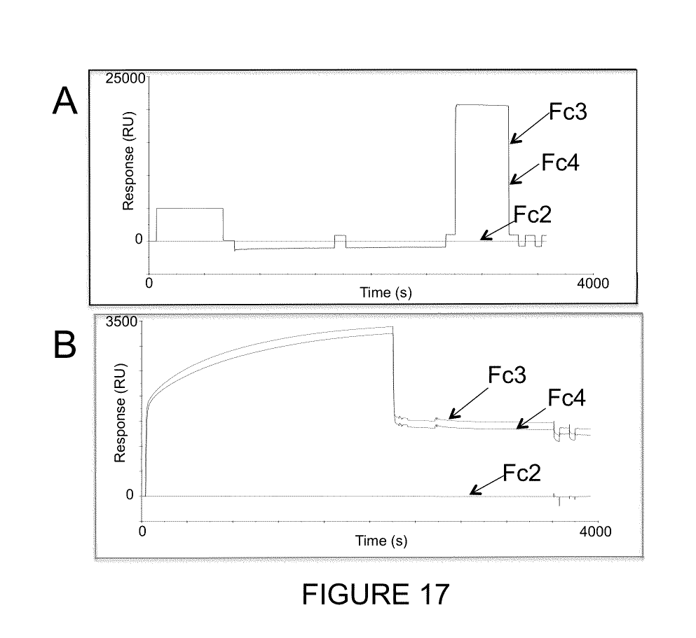

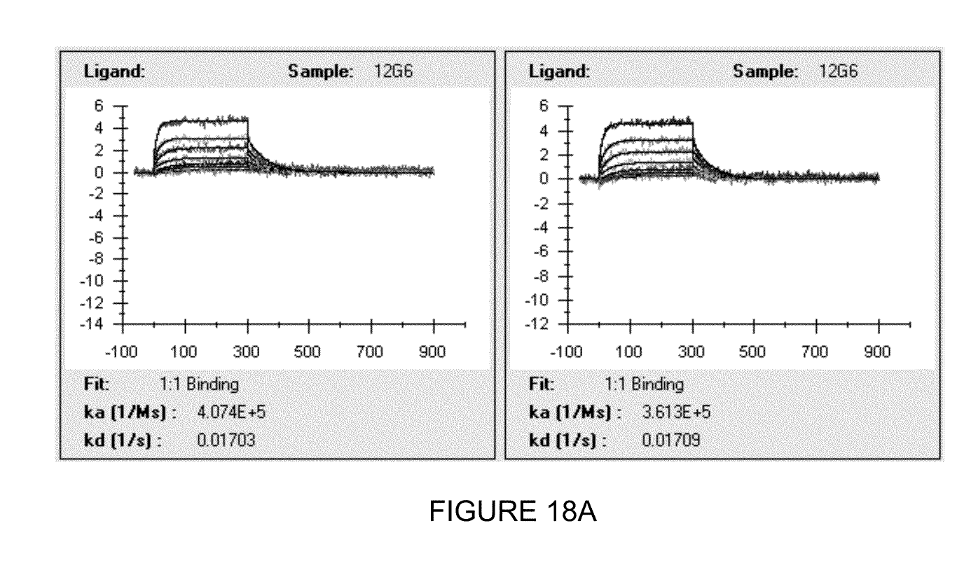

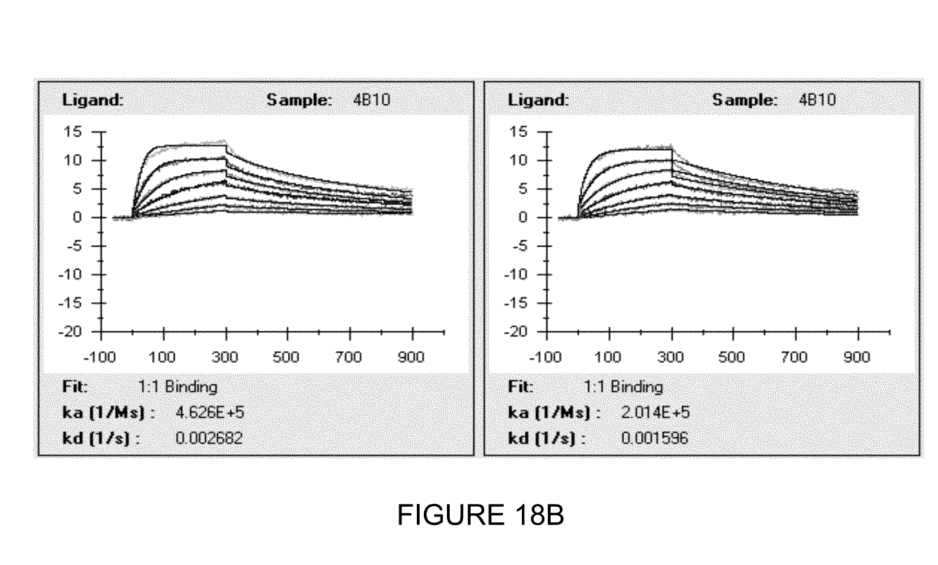

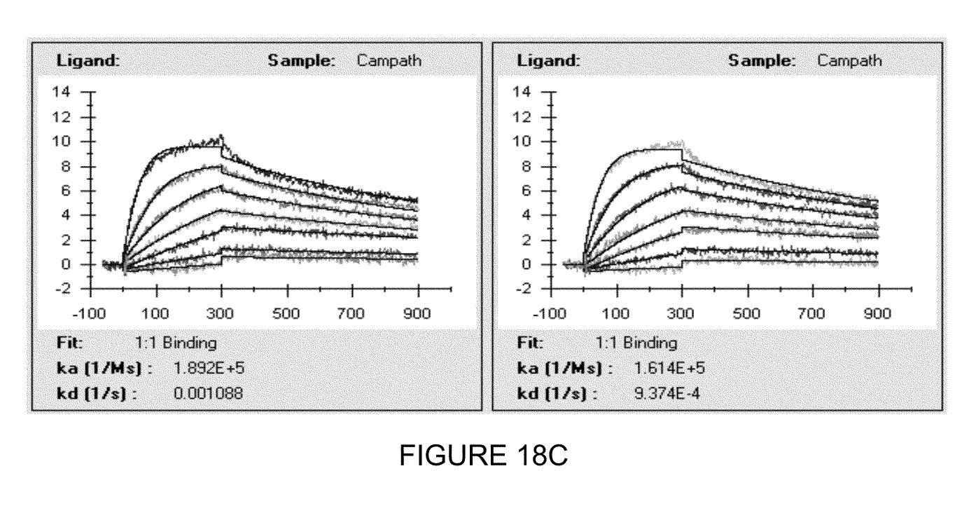

Since CD52 molecules were displayed on the VLP surface with native structure, VLPs were captured by using an anti-CD52 mAb (Campath) on the Biacore C1 sensor chip surface. As shown in FIG. 17, following activation of the C1 chip surface by standard NHS/EDC procedure, anti-CD52 antibody was immobilized identically on flow cell 3 (Fc3) and flow cell 4 (Fc4). CD52 VLPs were then captured by the anti-CD52 antibody at 1000 RU. The captured VLPs were further stabilized by crosslinking using 20 μM NHS/5 μM EDC for 7 min and excess crosslinkers were quenched by 1 M ethanolamine for 10 min. This limited chemical crosslinking step was sufficient to stabilize the VLP surface for subsequent anti-CD52 mAb kinetics assay as shown in FIG. 18. Reproducible kinetics sensorgrams were generated by both flow cells. Different mAb clones exhibited distinct Kₘ and K_{off} patterns, which were reflected by different kinetics and affinity data as shown in Table 5.

TABLE 5
Biacore kinetics of anti-CD52 mAbs on CD52 VLP surface
captured and stabilized on C1 chip surface

| Curve | ka (1/Ms) | kd (1/s) | KD (M) | Chi² (RU²) |
|---|---|---|---|---|
| 12G6 | 4.07E+05 | 1.70E−02 | 4.10E−08 | 0.0540089 |
| 12G6 2 | 3.61E+05 | 1.71E−02 | 4.73E−08 | 0.0608981 |
| 4B10 | 4.63E+05 | 2.68E−03 | 5.80E−09 | 0.123143 |
| 4B10 2 | 2.01E+05 | 1.60E−03 | 7.92E−09 | 0.1341931 |
| Campath | 1.89E+05 | 1.09E−03 | 5.75E−09 | 0.0534773 |
| Campath 2 | 1.61E+05 | 9.37E−04 | 5.81E−09 | 0.0537609 |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A method for immobilizing a membrane protein or membrane protein complex on an analytical surface, the method comprising:
(a) obtaining a membrane protein or membrane protein complex comprising a capture moiety;
(b) immobilizing the membrane protein or membrane protein complex on the analytical surface by means of the capture moiety; and
(c) stabilizing at least one of the secondary, tertiary, or quaternary structures of the immobilized membrane protein or membrane protein complex by crosslinking the immobilized membrane protein or membrane protein complex with a crosslinking reagent.

2. The method of claim 1, wherein the analytical surface is a surface plasmon resonance (SPR) surface.

3. The method of claim 1, wherein the capture moiety is a 6×His tag and the analytical surface is a nickel-nitrilotriacetic acid (Ni-NTA) surface.

4. The method of claim 1, wherein the crosslinking reagent forms a linkage between an amine moiety and a carboxylic acid moiety.

5. The method of claim 4, wherein the crosslinking reagent comprises:
(i) N-hydroxysulfosuccinimide (NHSS) and 1-ethyl-3-(3-dimethylaminopropyl)carbodiimide (EDC); or
(ii) N-hydroxysuccinimide (NHS) and 1-ethyl-3-(3-dimethylaminopropyl)carbodiimide (EDC).

6. The method of claim 1, wherein the crosslinking reagent forms a linkage between a thiol moiety and an amine moiety.

7. The method of claim 6, wherein the crosslinking reagent comprises:
(i) succinimidyl 4-(N-maleimidomethyl)cyclohexane-1-carboxylate (SMCC); or
(ii) 3-(2-pyridyldithio)propionate (SPDP).

8. The method of claim 1, wherein the crosslinking reagent forms a linkage between two amine moieties.

9. The method of claim 8, wherein the crosslinking reagent comprises bis(succinimidyl)penta(ethylene glycol) (BS(PEG)₅).

10. The method of claim 1, wherein the crosslinking reagent forms a linkage between two thiol moieties.

11. The method of claim 10, wherein the crosslinking reagent comprises:
(i) dithiobismaleimidoethane (DTME); or
(ii) 1,8-bismaleimido-diethyleneglycol (BM(PEG)₂).

12. The method of claim 1, wherein the crosslinking reagent forms a linkage between two carbohydrate moieties.

13. The method of claim 12, wherein the crosslinking reagent comprises:
(i) S-(2-thiopyridyl)-L-cysteine hydrazide (TPCH); or
(ii) S-(2-thiopyridyl)mercapto-propionohydrazide (TPMPH).

14. The method of claim 1, wherein the crosslinking reagent forms a linkage between a carbohydrate moiety and a thiol moiety.

15. The method of claim 14, wherein the crosslinking reagent comprises:
(i) N-beta-maleimidopropionic acid hydrazide (BMPH); or
(ii) 3-(2-pyridyldithio)propionyl hydrazide (PDPH).

16. (canceled)

17. A method for immobilizing a membrane protein or membrane protein complex on an analytical surface, the method comprising:
(a) obtaining a membrane protein or membrane protein complex modified with a capture moiety;
(b) coupling to the analytical surface an antibody specific for the capture moiety using a coupling reagent;
(c) immobilizing the membrane protein or membrane protein complex on the analytical surface by means of interaction between the antibody and the capture moiety; and
(d) stabilizing at least one of the secondary, tertiary, or quaternary structures of the immobilized membrane protein or membrane protein complex by crosslinking the immobilized membrane protein or membrane protein complex with a crosslinking reagent.

18. (canceled)

19. The method of claim 17, wherein the capture moiety is a 6×His tag and the antibody is an anti-6×His antibody.

20. The method of claim 17, wherein the membrane protein complex is a virus-like particle (VLP).

21-38. (canceled)

39. An analytical surface comprising:
a membrane protein or membrane protein complex comprising a capture moiety,
wherein the membrane protein or membrane protein complex is immobilized on the analytical surface by means of the capture moiety, and
wherein at least one of the secondary, tertiary, or quaternary structures of the membrane protein or membrane protein complex is stabilized by crosslinking.

40-54. (canceled)

55. An analytical surface comprising:
(a) a membrane protein or membrane protein complex comprising a capture moiety, and
(b) an antibody specific for the capture moiety,
wherein the antibody is coupled to the analytical surface using a coupling reagent,
wherein the membrane protein or membrane protein complex is immobilized on the analytical surface by means of interaction between the antibody and the capture moiety, and
wherein at least one of the secondary, tertiary, or quaternary structures of the membrane protein or membrane protein complex is stabilized by crosslinking.

56-76. (canceled)
